(19) United States
(12) Patent Application Publication
(10) Pub. No.: US 2022/0061919 A1
(43) Pub. Date: Mar. 3, 2022

(54) Method And Integrated System For Assisting In Setting Up A Personalized Therapeutic Approach For Patients Subject To Medical And Surgical Care

(71) Applicant: Abys Medical, La Rochelle (FR)

(72) Inventors: Arnaud DESTAINVILLE, Saint-Xandre (FR); Olivier RICHART, Le Bois-Plage-en-Re (FR); Sylvain ORDUREAU, Paris (FR)

(73) Assignee: Abys Medical, La Rochelle (FR)

(21) Appl. No.: 17/409,017

(22) Filed: Aug. 23, 2021

Related U.S. Application Data
(60) Provisional application No. 63/073,555, filed on Sep. 2, 2020.

(51) Int. Cl. A61B 34/10; G16H 70/60; G16H 50/50; A61B 34/00

(52) U.S. Cl. CPC A61B 34/10; G16H 70/60; G16H 50/50; A61B 2034/252; A61B 2034/102; A61B 2034/256; A61B 34/25

(57) ABSTRACT

A surgical support method including obtaining raw medical imaging data corresponding to a user from a remote computing architecture and reconstructing a digital model from the obtained raw medical imaging data. The digital model is two-dimensional or three-dimensional. The method includes determining at least one pathology based on the digital model, obtaining a sequence of surgical acts based on the at least one pathology, and generating a plurality of three-dimensional scenes by applying the sequence of surgical acts to the digital model.

### CROSS-REFERENCE TO RELATED APPLICATION

The application claims priority to U.S. provisional patent application Ser. No. 63/073,555 filed on Sep. 2, 2020, which is incorporated by reference herein.

### FIELD

The present disclosure relates to a method and integrated system for assisting in setting up a personalized therapeutic approach for patients subject to medical and surgical care.

### BACKGROUND

Operators or surgeons are faced with a need to perform an increasing number of medical procedures without technological assistance. For example, while many operators may perform surgeries with other healthcare workers physically assisting them, educational or training resources are not available during the surgery. Further, the healthcare industry is consistently facing a reduction in staff which is not only harmful to the operators but also to users or patients.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A surgical support method including obtaining raw medical imaging data corresponding to a user from a remote computing architecture and reconstructing a digital model from the obtained raw medical imaging data. The digital model is two-dimensional or three-dimensional. The method includes determining at least one pathology based on the digital model, obtaining a sequence of surgical acts based on the at least one pathology, and generating a plurality of three-dimensional scenes by applying the sequence of surgical acts to the digital model. The method includes simulating a virtual performance of the sequence of surgical acts on the digital model of the user using the generated plurality of three-dimensional scenes and displaying, on a display, at least one of: the reconstructed digital model, the at least one pathology, the generated plurality of three-dimensional scenes, and the simulated virtual performance.

In other aspects, the method includes extracting data of interest from the reconstructed digital model and determining the at least one pathology based on the extracted data of interest.

In other aspects, the method includes projecting the plurality of three-dimensional scenes onto the user to guide an operator. The projecting includes holographic projection.

In other aspects, the holographic projection can be manipulated by the operator or can be positioned on the user.

In other aspects, the generated plurality of three-dimensional scenes or the simulated virtual performance is displayed in a collaborative mode to remotely assist the operator to perform a corresponding procedure requiring multiple assessments or to train the operator in an observational mode.

In other aspects, the method includes implementing artificial intelligence and/or a simulation of physical systems by numerical mathematic modeling to model the simulated virtual performance.

In other aspects, the method includes generating device digital models of at least one of: anatomical elements, implantable medical devices, and ancillary instrumentation corresponding to the sequence of surgical acts. In other aspects, the device digital models include three-dimensional objects based on the reconstructed digital model or data of interest extracted from the reconstructed digital model.

In other aspects, the anatomical elements, the implantable medical devices, or the ancillary instrumentation are based on anatomy of the user.

In other aspects, the method includes projecting the device digital models onto the user to guide an operator. In other aspects, the projecting includes holographic projection.

In other aspects, the method includes, in response to a structured surgical planning of the user ending, generating device digital models of at least one of: anatomical elements, implantable medical devices, and ancillary instrumentation.

In other aspects, the method includes generating a digital file based on the device digital models and transmitting the generated digital file to a manufacturing facility to create a three-dimensional model using the generated digital file.

A surgical support system includes at least one processor and a memory coupled to the at least one processor. The memory stores a database including a set of raw medical imaging data and a set of surgical acts. Each set of raw medical imaging data corresponds to a user. The memory stores instructions executed by the at least one processor and the instructions include obtaining raw medical imaging data corresponding to a user from the database. The instructions include reconstructing a digital model from the obtained raw medical imaging data. The digital model is two-dimensional or three-dimensional. The instructions include extracting data of interest from the reconstructed digital model, determining at least one pathology based on the extracted data of interest, and obtaining a sequence of surgical acts based on the at least one pathology from the database. The instructions include generating a plurality of three-dimensional scenes by applying the sequence of surgical acts to the extracted data of interest, simulating a virtual performance of the sequence of surgical acts on the digital model of the user using the generated plurality of three-dimensional scenes, and displaying, on a display, at least one of: the reconstructed digital model, the at least one pathology, the generated plurality of three-dimensional scenes, and the simulated virtual performance.

In other aspects, the instructions include projecting the plurality of three-dimensional scenes onto the user to guide an operator. The projecting includes holographic projection.

In other aspects, the holographic projection can be manipulated by the operator or can be positioned on the user.

In other aspects, the generated plurality of three-dimensional scenes or the simulated virtual performance is displayed in a collaborative mode to remotely assist the operator to perform a corresponding procedure requiring multiple assessments or to train the operator in an observational mode.

In other aspects, the instructions include implementing artificial intelligence and/or a simulation of physical systems by numerical mathematic modeling to model the simulated virtual performance.

In other aspects, the instructions include generating device digital models of at least one of: anatomical elements, implantable medical devices, and ancillary instrumentation corresponding to the sequence of surgical acts. In other aspects, the device digital models include three-dimensional objects based on the reconstructed digital model or the extracted data of interest.

In other aspects, the anatomical elements, the implantable medical devices, or the ancillary instrumentation are based on anatomy of the user.

In other aspects, the instructions include projecting the device digital models onto the user to guide an operator. The projecting includes holographic projection.

Computer software, stored in a non-transitory computer-readable memory including instructions receiving medical imaging data corresponding to a user from a database and instructions creating a digital model from the medical imaging data. The instructions include using data of interest from the digital model, determining at least one pathology based on the data of interest, and receiving a sequence of surgical acts based on the at least one pathology. The instructions include generating a visual image by applying the sequence of surgical acts to the data of interest and simulating a virtual performance of the sequence of surgical acts on the digital model of the user using the generated images. The instructions include projecting, via a headset, the generated images to guide an operator and displaying, on a display of the headset, at least one of: the digital model, the at least one pathology, the generated images, and the simulated virtual performance.

In other aspects, projecting includes projecting a holographic projection, and the holographic projection can be manipulated by the operator or can be positioned on the user.

In other aspects, the generated images or the simulated virtual performance is displayed in a collaborative mode to remotely assist the operator to perform a corresponding procedure requiring multiple assessments or to train the operator in an observational mode.

In other aspects, the instructions include implementing artificial intelligence and/or a simulation of physical systems by numerical mathematic modeling to model the simulated virtual performance.

In other aspects, the instructions include generating device digital models of at least one of: anatomical elements, implantable medical devices, and ancillary instrumentation corresponding to the sequence of surgical acts, wherein the device digital models include three-dimensional objects based on the reconstructed digital model or the data of interest.

In other aspects, the anatomical elements, the implantable medical devices, or the ancillary instrumentation are based on anatomy of the user.

In other aspects, the instructions include projecting the device digital models onto the user to guide the operator. The projecting includes holographic projection.

A method of using for surgical preparation and surgery includes obtaining medical imaging data corresponding to a user, constructing a digital model from the obtained medical imaging data, and selecting a sequence of surgical acts from a database. The method includes generating a plurality of scenes by applying the sequence of surgical acts to the digital model, simulating a virtual performance of the sequence of surgical acts on the digital model of the user using the generated scenes, and displaying at least one of: the reconstructed digital model, the generated plurality of scenes, and the simulated virtual performance. The method includes showing the plurality of scenes to guide surgical preparation and surgery associated with the user.

In other aspects, the method includes extracting data of interest from the reconstructed digital model and determining at least one pathology based on the extracted data of interest.

In other aspects, the method includes showing includes projecting a holographic projection, and the holographic projection can be manipulated by the operator or can be positioned on the user.

In other aspects, the method includes implementing artificial intelligence and/or a simulation of physical systems by numerical mathematic modeling to model the simulated virtual performance.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a high-level functional block diagram of an embodiment of a system according to the present invention.

FIG. 2 is a high-level functional block diagram of a data processing infrastructure of an embodiment of a system according to the present invention.

FIG. 3 is an example functional block diagram of the computing architecture.

FIG. 4 is an example function block diagram of the processing and algorithms module.

FIG. 5 is an example functional block diagram of the data processing and display module.

FIG. 6 is a functional block diagram depicting the connections between the various modules of the system.

FIG. 7 is a flowchart depicting the steps of surgical planning, surgical guidance, and corresponding streaming.

FIG. 8 is another example flowchart describing the specific implementation steps of surgical planning.

FIG. 9 is another example flowchart describing the specific implementation steps of surgical guidance.

FIG. 10 is a high level depiction of an implementation of the system.

FIG. 11 is a graphical depiction of an operator view of a guided surgical operation using the system.

In the drawings, reference numbers may be reused to identify similar and/or identical elements.

### DETAILED DESCRIPTION

The presented system describes a surgical planning and guidance system for assistance in performing, training, and following a series of steps corresponding to a particular surgery, such an implanting a device into a user.

### Definitions

API: In computing, an application programming interface is a standardized set of classes, methods, functions, and constants that serves as the facade through which software offers services to other software.

CPU: A processor (or Central Processing Unit) is a component present in many electronic devices that executes the machine instructions of computer programs.

Medical device: according to the World Health Organization (“WHO Global Model Regulatory Framework for Medical Devices including in vitro diagnostic medical devices”, World Health Organization Medical device technical series, page 8, 2019), a medical device means any instrument, apparatus, implement, machine, implant, reagent for in vitro use, software, material or other similar or related article, intended by the manufacturer to be used, alone or in combination, for human beings, for one or more of the specific medical purpose(s) of: (i) diagnosis, prevention, monitoring, treatment or alleviation of disease; (ii) diagnosis, monitoring, treatment, alleviation of or compensation for an injury; (iii) investigation, replacement, modification or support of the anatomy or of a physiological process, (iv) supporting or sustaining life; (v) control of conception; (vi) disinfection of medical devices; or (vii) providing information by means of in vitro examination of specimens derived from the human body; and in each case does not achieve its primary intended action by pharmacological, immunological or metabolic means, in or on the human body, but which may be assisted in its intended function by such means. Certain additional products may be considered medical devices by some regulations, including: (i) disinfection substances; (ii) aids for persons with disabilities; (iii) devices incorporating animal and/or human tissues; or (iii) devices for in vitro fertilization or assisted reproduction technologies. By extension, these definitions are considered to be applicable to animal medicine for the present invention;

Flops (Floating-point operations per second): Measurement of the speed of a processor or one of the arithmetic calculation units of a processor by the number of floating-point operations that can be performed per second;

GPU: A graphics processor, or GPU (Graphics Processing Unit) is an integrated circuit providing the calculation functions of the display;

Teraflops: One teraflop corresponds to 1012 flops.

### Technical Problems Addressed by the Invention

The lengthening of the average lifespan of the world's population is a major cause calling for extensive health action to improve the efficiency of patient care processes. Indeed, this growing use of medicine is occurring under conditions of increasingly contrasting imbalance between the number of patients and the number of health professionals, the former increasing more significantly than the latter. When applied to surgery, this problem becomes significant, because this discipline of medicine involves, on the one hand, organization and substantial, specific and costly resources, and on the other hand, is potentially high risk for each of the parties. These risks are either of a medical nature, going as far as lethality in an extreme—but real—case for the patient, or involve criminal and financial liability for the practitioner as well as the healthcare establishment that provides the care (private or public structure).

The handling of this issue requires a multiaxial analytical approach: analysis of the patient's current care cycle, analysis of the growing role of surgical planning, analysis of the growing role of personalized therapy in the surgical act, analysis of the relevance current technological solutions and, finally, the solution into which the present invention is projected as an emanation of industry 4.0.

This methodology will shed light on the complex and interrelated problems that the invention addresses. It will then be illustrated, in a non-limiting approach, with respect to the surgical specialty chosen for its emblematic character: orthopedic and trauma surgery.

### Analysis of the Patient Care Cycle in the Current Context

With a view to deploying an ever-improving healthcare offering for patients, fully meeting the expectations of surgical professionals requires combining an acute knowledge of healthcare establishments and their functioning within state healthcare systems, digital technologies applied to health, and finally the development of medical devices in accordance with applicable health regulations. When applied to the course of care, however, the act of surgery is only one stage of the journey. Therapeutic care can in fact be broken down into four main phases.

The diagnostic phase: after a prior medical consultation, the suffering patient is referred to a specialist, at the same time as he is prescribed medical examinations meant to provide the specialist doctor with sufficient information about the nature and extent of the pathology at the root of the suffering in order to make a diagnosis. A very large majority of these examinations are of a biological and imaging nature, the typology of which varies based on diagnostic needs (computed tomography, angiography, radiology, magnetic resonance imaging, etc.). Imaging examinations are done by a radiologist, who therefore proceeds, based on two-dimensional (abbreviated by the term 2D throughout this document) or three-dimensional (abbreviated by the term 3D throughout this document) data produced for this patient, with the diagnosis that will be sent to the prescribing physician as well as to the specialist doctor. The specialist doctor, depending on his specialty and the results of medical examinations, may or may not decide on a therapeutic approach involving surgery for this patient. If he decides to intervene surgically, the process enters a phase called “preoperative phase”.

The preoperative phase: once the decision to operate on the patient has been made, the specialist doctor initiates a preparation process for the operation. The first theoretical step consists of carrying out the planning of the surgery in question, consisting of technically structuring the possible steps: surgical approach to optimize access to the operating site, choice of ancillary instrumentation or assistance equipment to facilitate or perform some or all of the operating procedures (forceps, bone holding forceps, surgical guides, cutting tools, camera, laparoscopic equipment, robotic arm, etc.), choice of implantable devices if applicable (prosthetic net, osteoarticular hip or knee prosthesis, vascular endoprosthesis, osteosynthesis material for fracture, pacemaker, etc.), analysis and measurement via the data available in imaging, etc. Surgical planning therefore consists first of all in simulating, by whatever means, the surgery to be performed: on paper, by computer tool, or even informally by experience without any particular support. The second stage of this phase follows from the first and is operational: consultation with the teams in charge of the operating suites to check the availability of the necessary equipment in stock or whether it must be ordered/reordered to allow the surgery to be scheduled. The equipment specific to the anatomy of the patient in question that may be required by certain surgical procedures is ordered at this stage. In this case, the devices are said to be “tailored” or “specific”, implantable or ancillary (for example in case of unusual morphology). The entire preoperative process is also done in consultation with the other medical disciplines that may be involved in the medical care process, such as anesthetic medicine for example.

The intraoperative phase: this phase consists of performing the actual surgery on the patient according to the technical and operational preparations validated and programmed during the two preceding stages. The surgeon proceeds according to his plan with the identified equipment, which will have been prepared by the teams affiliated with the operating room and/or by outside service providers for the procedure (order, inspection, assembly, verification, sterilization, etc.). It is important to specify that different and accelerated arrangements can be made for phases 1 to 3 in urgent cases where the patient's survival is in question (so-called “hot” surgery, as opposed to so-called “cold” surgery, which can be scheduled). The choice of accelerated care followed by a short hospital stay can also be based on budget optimization goals in order to reduce the costs of therapeutic treatments.

The postoperative phase: this phase incorporates care from when the patient wakes up until he is discharged from the establishment, including intermediate phases which may for example include care, monitoring, or even one or more additional or complementary procedure(s). A growing number of procedures are now performed on an outpatient basis and do not require a post-surgery hospital stay. The patient monitoring and rehabilitation protocols are defined during this phase, if applicable.

### Analysis of the Growing Role of Surgical Planning as a Driver of Performance and Safety for Patient Care

In view of the intensification of the demand for care, the imbalance between the number of patients and the number of surgeons is destabilizing a chain, which may become a series of bottlenecks. The situation today is resulting in a greatly increased capacity requirement for each actor in the process (radiologists and surgeons first, but also nurses, operating room staff, anesthetists, etc.), at the risk of sacrificing quality for numbers. While a decreasing performance resulting from an excessive need which is difficult to curb and disproportionate to the available means is to be avoided in any service activity, in matters of health it is a major risk that must be eliminated.

The demand for imaging services is thus constantly increasing (systematization of the prevention, diagnosis and follow-up stages), exceeding the supply of qualified radiologists. Technological progress in equipment now makes it possible to obtain several hundred images per examination, which can result for a single radiologist in an order to process several tens of thousands of images per day, which is incompatible with human capacity without compromising the quality of the diagnosis.

In this highly constrained context, using the power of IT to speed up processing and analyses, facilitate diagnoses and make them more reliable, secure the transmission of information between different players in the therapeutic chain or even increase surgical capacities is a rapidly growing process, to the point that the digitization of the patient care journey must be considered an unprecedented technological revolution. As part of this transformation, the digital conversion of a growing number of healthcare procedures is a major issue that is gradually structuring this paradigm shift for medical teams with the aim of providing ever more and better care.

At the heart of this transformation is the crux of the therapeutic care journey made up of multiple phases, including: (1) Diagnostic actions, (2) Preoperative actions, and (3) Intraoperative actions.

This node concentrates the issues of medical performance (therapeutic outcome) and operational performance (organizational fluidity, financial burden), as well as health safety issues (exposure to risks). All medical specialties face these problems, in all countries where structured health systems exist.

One of the preferred solutions today to address part of the problem is the adoption of artificial intelligence technologies that should make it possible to rapidly exploit the colossal amount of information made available by medical image production systems, and therefore improve the capabilities of radiologists by automating diagnostic assistance systems in certain disciplines. In addition to this action dedicated to diagnostic phase 1, a second action, complementary to the first, consists of giving the surgeon an increased ability to use medical imaging data to strengthen the surgical planning stage. Without replacing radiologists, this approach has the strong interest of increasing knowledge of the pathological case to be treated by the surgeon, with the prospect of optimal preparation of the operation for the benefit of the patient. Since the recent beginnings of additive manufacturing, the growing integration of a structured approach to surgical planning on simple modes such as 3D printing using polymer or plaster of the organ(s) to be operated on for use by the surgeon has shown, within a still-emerging literature, a reduction in the duration of the surgical procedures in question.

This reduction must be correlated with better preparation, and therefore knowledge, of the operating site, before surgical opening, as well as with modifications of the therapeutic approach that may be decided upon by the surgeon due to the contribution of new data. Sometimes drastic reductions have thus been reported in the expected duration of the surgery in the event of a radical change in the initial procedure strategy. This reduction in the duration of the procedure has several consequences on the therapeutic and operational levels. On the one hand, it makes it possible to reduce the patient's exposure to the risks inherent to the procedure: nosocomial infection, blood transfusion requirements or even anesthesia. Apart from the objective of avoiding liability as much as possible for damage to health suffered by a patient who considers himself injured as a result of a procedure undergone within their structures, hospitals also have an operational and financial interest in this reduction in operating time: they can increase the daily number of surgeries, and therefore their profitability.

### An Increasingly Personalized Therapy

Therapeutic personalization in surgery is defined as the taking into account of the physiological, morphological or anatomical specificities of a patient as input data for the preparation and performance of a procedure, with the aim of adapting a medical protocol to the patient's particular case. The objective is twofold, with an equivalent benefit/risk ratio: better surgical performance for stable or reduced operational costs.

In the context of an instrumented procedure possibly requiring the use of one or more implantable device(s) and/or ancillary instrumentation specific to the patient, the operational gain described in the previous paragraph can be further increased. Beyond the performance contribution, this therapeutic choice makes it possible to optimize the operating results of health institutes by reducing inventories of devices within the establishments and by reducing operational management requirements (unit traceability, sterilization, inspection, etc.).

This personalized approach, which has existed for several years for certain medical specialties (orthopedic surgery, for example), nevertheless suffers from an application that remains exceptional. The origin of this accessory nature is twofold.

The first is linked to the implementation between the surgeon and the manufacturers of planning/manufacturing process devices that are often unsuitable for unit production and therefore expensive and restrictive for the parties (use of equipment normally due for mass production, analysis and design outsourced to dedicated third-party teams).

The second origin is a direct consequence of the first: due to the high cost of custom-made devices according to the method described in the previous paragraph, the majority of health regulations persist in a requirement of exceptional use which must be justified by a medical prescription. Because the inertia of the regulatory texts prevails, the persistence of the initial model is logically implied.

Although a strong trend towards dual surgeon/engineer training is emerging within the medical profession (designated by the neologism “surgineer”, the combination of “surgeon” and “engineer”), it remains rare for the moment and the role of a third party remains preponderant at several levels: the recovery of patient files (loading by the practitioner on a remote server or by sending to the third party of a digital storage medium such as a CD-ROM, for example), 3D reconstruction, preparation for modeling (for example: segmentation of the 3D file to extract specific tissues or the skeleton of an organism, for example), software knowledge, methodology for understanding 3D, computing power locally adapted to process information and carry out preliminary modeling which will be reviewed by the surgeon, etc.

With the advent of the additive manufacturing process, and more broadly due to the increasing digitization of patient care cycles already mentioned in this document, the linearization of the manufacturing processes for patient-specific medical devices is nevertheless underway, with a regulatory dynamic that is gradually tending to integrate this powerful development within legal requirements. The linearization of the chaining of links between diagnosis (phase 1) and surgery (phase 3) implies, however, fully mastering the second essential link, which is operative planning, and in particular the reality of the software architecture that must support it in order to make it compatible with all performance, safety and cost requirements.

### Analysis of the Relevance of Current Technological Solutions

The support given to the analysis activity in medical imaging by artificial intelligence algorithms therefore increases, for certain specialties and certain examinations, the capacities of the radiologist and secures the diagnosis by avoiding congestion of imaging services. As previously mentioned, currently there is little systematization of the direct chaining with planning of the surgical procedure that may be validated after the diagnosis, outside of complex surgeries presenting a high risk for the patient (for example: neurosurgery, cardiology, oncology). With a view to generalizing the preoperative preparation activity to all surgical specialties, it is therefore critical to be able to develop solutions that allow this linear chain between the imaging examination and the performance of surgery through planning as a gateway to surgical assistance (the manufacturing of medical devices, implantable or ancillary, modeled on a custom basis by the surgeon as well as assistance by surgical guidance).

The most direct solution is therefore to transfer part of the diagnostic analysis into the hands of the practitioners themselves by assisting them with software adapted to their objectives. The added value for surgeons is therefore less in visualization and use for personalized diagnostic purposes than in supplementing this preliminary work with software functionalities that make it possible to model the stages of surgery in their entirety. This approach has already been implemented in commercial solutions that meet the business objective of planning, some also allow a link to be made with external computerized surgical assistance systems, or even making it possible to model implantable devices or ancillary instrumentation specific to the patient's anatomy. All of these products find their justification in the digital automation dynamics of phases 1 to 3.

The question of the business response is not, however, the only response to be provided, because if we wish to generalize the chaining of diagnostic/modeling/surgical assistance solutions, it is fundamental to ask the question of the technical implementation in coherence with the constraints of the entire health ecosystem. A medical device must indeed present a favorable benefit/risk ratio, but also provide surgeons with a reliable, efficient, secure and economically acceptable tool. In the context of using increasingly massive datasets in support of the activities of phases 1 to 3, it is therefore a question of fully understanding the IT architectural issue behind the business issue of providing medical devices to treat a given pathology.

An analysis of the technical principles underpinning current solutions makes it possible to highlight certain limitations linked to technical choices that are hardly compatible with lasting resistance to the test of time.

### The Availability of Computing Performance at the Heart of the Problem

The preferred medical imaging files to date for 3D modeling during surgical planning are those obtained by computer-assisted tomodensitometry, or even cartographic files obtained by Magnetic Resonance Imaging. Although generated by different acquisition methods, the files obtained are encoded in a standardized format called DICOM (acronym for Digital Imaging and Communication in Medicine). These files, which contain a large amount of information relating to the three-dimensional structuring of the analysis object, are large files (several hundred megabytes) which require high computing power to be used in their raw state. The computed tomography acquisition method for example makes it possible to reconstruct, from series of two-dimensional slices obtained by measurements taken outside an object, and by stacking these measurements, three-dimensional volumes containing all of the information detectable by the device as a function of its precision and the acquisition program.

The basic units of these reconstituted volumes are called “voxels” in reference to their status as the smallest discretionary solid element in a three-dimensional scene. Since the degree of precision of an analysis is directly correlated with the definition of the object of said analysis, the precision of a diagnosis is therefore logically directly dependent, with equivalent calibration, on the number of voxels per unit of volume, therefore on the resolution. The higher the resolution, the more faithful the 3D reconstruction will be to the analyzed object, nevertheless leading to the delivery of increasingly massive files at an equivalent volume. The expectation of reliability and high performance, in terms of both diagnostics surgical planning, combined with increasingly precise and efficient acquisition systems, therefore implies the availability for the concerned actors of a strong computing power within the patient care chain.

The reality today is that the technical proposals aiming to support this chaining (with modeling of medical devices that may or may not be specific to the anatomical morphology of a patient) struggle to meet all of the requirements of the health specifications. The pitfall essentially relates to the methods of achieving performance for each link in the chain, which can follow several paths, the specificities of which should be examined carefully.

The most obvious way to reduce computational requirements is to simplify the model that will serve as the basis for the entire process. The amount of data in a file can be reduced by applying a compression algorithm that will select sub-lots of data according to a predefined setting. DICOM volume files are compressed by converting the volume data in voxels into area data via a mesh action, of the object of interest, commonly referred to as “mesh”. The rendering of said object is thus simplified into a three-dimensional object presenting only the envelopes of the initial volume, the envelopes being made up of vertices, edges and faces. This algorithmic action thus makes it possible to reduce the size of the files and to facilitate their handling in order to downwardly revise the computing power requirements.

This therefore amounts to considerably reducing the quantity of information contained within these envelopes even before having carried out any manipulation, in particular the second step common to all surgical planning: segmentation. Segmentation in fact consists of applying a computer algorithm to 2D images or to the volume reconstructed in 3D from these images in order to display and/or keep only certain specific data, chosen according to predefined criteria. This may for example consist of keeping, within a tomodensitometric examination, only one type of tissue or anatomical element depending on the medical specialty and the objective (bone tissue or individual bone for orthopedic or maxillofacial surgery, blood vessels for vascular surgery, liver for liver surgery, etc.). The quality of the material that will be used following the planning operations can therefore be greatly affected by the application of this algorithm on an already compressed file, with already reduced fidelity compared to the reconstruction resulting from the original DICOM.

The choice of the resolution of the surface mesh determining the compression rate of the file upstream of the segmentation, the mathematical modeling is therefore critical and, under certain thresholds, casts doubt on the fidelity of the rendering for diagnostic or planning use, therefore for modeling of the stages of surgery in preparation for actual therapy in a patient. The model runs the risk of representing the reality of its patient insufficiently, which does not appear to be sustainable in a health ecosystem where performance, and therefore faithfulness of the model to reality, must become the standard with regard to the technologies available today.

Due to the computational requirements inherent to the handling of voxels, the choice of simplifying data by meshing is generalized: prior visualization by the surgeon comparable to a complement to radiological diagnosis (soft tissue surgery: neurosurgery, vascular or hepatological surgery, etc.), modeling of ancillary surgical assistance instruments specific to the patient's morphology (guides in orthopedic surgery: positioning, drilling, cutting, orientation, etc.) or even decision support by allowing the surgeon to choose a model of implants within a library of preconceived solid elements to superimpose them on the anatomical object in 3D.

### Solutions Developed Natively in the Cloud as a Response to Dissatisfaction

The provision of high performance computing for the manipulation of voxel data in 3D or simply to move from a software model installed locally on the operator's computers—and which is therefore limited to the performance of these machines and the need for development specific to the computer operating system (Windows®, MacOS®, etc.)—to an Internet platform-type model for the mobilization of remote computer servers allowing the transfer of this computation in addition to the mass storage of data, is currently growing in the medical field, which consumes large quantities of 3D data for the reasons set out above. This growth is thus logically supported by the marketing of services for the provision of “Cloud” or “Cloud computing” solutions (generic term used in many languages, including French-speaking countries, which may sometimes prefer “solution nuagique”) dedicated to either remote data storage or high performance graphics computing. These services allow access to remote computing machines equipped with high graphics computing power through the concentration of graphics processors (acronym for Graphics Processing Unit). The company Microsoft®, which is associated with the designer and manufacturer of NVIDIA® graphics cards, is for example currently a major player in providing these solutions through its Microsoft Azure® product.

The companies that offer computerized surgical planning products using Cloud Computing to linearize the chaining between phase 1 (radiological diagnosis) and phase 2 (preparation for surgery) are mainly positioned on two services that can be individual or combined: (1) a direct link with management systems for medical files and (2) the surgeon using the file via a web browser.

In other words, the first option is a direct link with management systems for medical imaging files managed by radiologists, and designated by the acronym PACS (Picture Archiving Communication System) to store files on a new server to then be processed by a third party for surgical planning preparation activities.

Additionally, the second option is the possibility for the surgeon to use the file in question using software that is no longer run locally on the practitioner's computer but operates on a website accessible via a web browser such as Google™ Chrome™, Microsoft® Edge, Mozilla Firefox® or Opera™.

This second option, which is very attractive, is logically in high demand with regard to the desired objective. Today, however, there are software offers on the market to aid diagnosis/surgical planning which suffer from several major pitfalls before they can be sustainable.

Data security: health data is now subject to strict controls and must be hosted on certified servers, which does not prevent a certain opacity in the control of data duplication. This is particularly true during the transfer and transit phases, when these data undergo several successive algorithmic processing operations that require intermediate records, and therefore storage.

Technical restrictions: several issues are emerging today for the use of a solution based on the use of Cloud Computing, with the common origin not of the construction of integrated solutions, but rather the sometimes forced arrangement of technological components that were not initially developed for these purpose.

Real-time rendering of 3D scenes: the objective being to allow a surgeon to carry out advanced 3D planning combining simple visualization of anatomical data with complex models of surgical steps, the rendering must be faithful, fluid and of good quality for all operators and observers of a scene and not dependent on the performance of the computer terminal (computer, tablet, smartphone, virtual or augmented reality visualization system, etc.), which is the most common situation and also explains the use of data simplification.

3D visualization engines: the initial path followed by industries to meet needs in terms of interactive 3D visualization is the diversion of 3D engines from video games, a discipline that is very demanding in terms of performance. As efficient as these engines are in video games, this efficiency is not, however, obtained by simple transposition for use in other fields, particularly in association with cloud computing technology. They simply weren't designed with these needs in mind, and, without upgrades, are not solutions that will stand the test of time.

The collaborative approach: one of the major interests of cloud computing is the sharing of data in real time. In the case of surgery, in particular from the perspective of surgical planning or remote assessment activities carried out by a group of surgeons or doctors, it is essential to offer an accessible and efficient technology for sharing a scene in 3D. Today, it is a cornerstone in the expansion of solutions based on the cloud computing GPU, the performance of which is a combination of quality and availability of infrastructure (Cloud GPU, telecommunications networks, operator/user equipment) and choice of software architecture to support these solutions with as low a latency as possible (latency refers to the time required for a packet of computer data to travel from source to destination over a network).

Historically, and faced with the absence of solutions developed to exploit cloud computing technology natively, the preferred path has been to adapt the applications by designing them to exploit the hardware resources of the computer terminal, to then bring them to the cloud via virtual machines that are lastly synchronized in order to visualize the same three-dimensional scene (for example: a heart reconstructed in 3D obtained by MRI). A virtual machine is an illusion of a computer machine created by emulation software that simulates the presence of hardware resources. The problem encountered is that the software ported to the cloud is not developed to make optimal use of cloud functionality (this would require colossal reprogramming work to match the requirements of GPU technologies), thus posing not only synchronization problems, but also the problem of high operating costs by requiring the deployment of a virtual machine with one GPU per operator and per connection.

This synthetic analysis of the situation shows that although the product offering is substantial, the offering of real solutions allowing the use of cloud computing technologies to support the fluidification of the diagnostic/surgical planning chain while respecting operator expectations, reimbursement systems and health regulations today seems largely unsatisfactory. Running a cloud-based solution is not simply about transporting the initial solution in a container in order to run in a leased virtual machine on remote servers. It is in this context that new solutions, designed natively for the exploitation of the Cloud GPU through an innovative and complex architecture allowing the shared distribution of 3D scenes. The pioneering solution in this area, natively exploiting the Cloud GPU since it entered the market in 2017, is the collaborative platform in the form of a cloud-type farm for 3D rendering and the calculation of 3DVerse® algorithms (www.3dverse.com). This solution thus acts as the first 3D engine purposefully structured via cloud computing, replacing the model requiring the resources of individualized devices. The deployment of this new type of solution thus constitutes a favorable base for the implementation of cross-cutting projects such as the linearization of solutions in support of the digitization of surgical patient care, subject to the development of innovative communication applications and models that are compatible with international regulatory requirements applicable to medical devices.

As previously mentioned, before addressing business issues of a surgical nature, we must ask ourselves about the technological structuring that allows the implementation of an “all-in-one” solution at the level of the surgeon. This implementation has so far been described through applications of diagnostic assistance (phase 1), surgical planning (phase 2) and finally assistance in the modeling of implantable medical devices or ancillary instrumentation for assistance with the surgical procedure (phase 3). The corollary of this route dedicated to personalized therapy, however, is the need to provide the surgeon with sufficient information allowing him to implant and/or use specific instrumentation with maximum safety, and therefore to guide his surgical procedure. The two historical routes are computer-assisted surgery (also referred to as surgical “navigation”) and surgical robotics, both of which are used and are usable from a surgical planning phase that concentrates all the considerations mentioned thus far on it. Depending on their degree of complexity and autonomy, these systems can integrate artificial intelligence algorithms, mainly for anatomical recognition. With a view to a wider use of surgical planning and a less demanding implementation in the operating room than required by navigation stations or robots, a third avenue has naturally opened up as an aid to the surgical procedure: augmented reality.

### Holographic Assistance to Serve the Surgeon

In terms of assistance with the surgical procedure, the main route taken for several years has been that of so-called “computer-assisted” surgeries, which allow the surgeon to assist and monitor his procedure using computer systems. We then speak of surgical “navigation” or “guidance”, because the principle of these systems is to allow the surgeon to follow all or part of a validated preoperative plan by comparing it in real time using specific data measured on the patient during the surgical operation. Osteoarticular prosthetic surgery (hip, knee, spine, etc.) thus saw the birth of this technology more than 30 years ago, resulting in interactive assistance for navigation stations, which for example operate on the principle of the detection by sensors (possibly of the infrared type) of fixed anatomical landmarks determined by the surgeon initially allowing him to calibrate the spatial positions of the “elements” of the scene: patient, surgeon's instruments, anatomical elements. Secondly, the system allows the surgeon to record the patient's anatomical data in 3D by palpation using a specific tactile instrument, possibly detected by the same sensors.

The system having recorded the spatial coordinates of the fixed reference frames, it is thus able to reconstitute the areas treated by the surgeon dynamically, then to compare them with the initial planning carried out upstream of the procedure and to assist in the decision-making phase centered mainly on the dimensional choices of implantable medical devices (total knee prosthesis components, for example). This example can be extended to other specialties such as neurosurgery or cardiac surgery; the technologies used for detection may vary depending on the manufacturer of the stations in question (QR code, for example). The original examinations can also be different (Magnetic Resonance Imaging, computed tomography angiography, etc.), but the principles remain comparable. One of the approaches being developed to improve the performance of this surgical guidance involves the use of a technology of the future: augmented reality, also called mixed reality. For convenience, only the term “augmented reality” will be used in the remainder of the document.

Augmented reality is defined as the superposition of reality and elements calculated by a computer system in real time. These elements can be of multiple natures: sound, videographic, haptic, two-dimensional, three-dimensional, etc. Widely known for film or video games, the increasing reliability of support technologies has opened up demanding and often highly regulated professional paths to augmented reality, such as industry or the medical sector. Although the attractiveness of these fields is initially the direct result of the amazement of first-time operators in the face of a technology that was still recently categorized as fictional, this is no longer the case today. Indeed, the reliability of visualization equipment in augmented reality (example: Microsoft® HoloLens®), combined with the prospect of improved performance and profitability, have now resulted in the existence of many usage scenarios of interest, such as technical inspection in industry or assistance with surgery in the medical sector.

These visualization systems consist of individual devices, possibly in the form of helmets, masks, glasses, and more generally any systems composed of a frame resting on the head (such as a headset) while being held by the head (ears, nose, skull, etc.) making it possible to visualize, through neutral or adaptive optical lenses or projection on said lenses or via screens, 3D scenes made up of holograms juxtaposed with reality. Unlike augmented reality, the principle of virtual reality is total immersion of the person equipped with a visualization system within a scene calculated in real time by a computer system, and therefore cannot be used in patient intervention situations. Virtual reality can, however, be a technology perfectly suited to surgical planning.

In comparison with computer-assisted surgery stations, augmented reality presents a high potential value added for surgical guidance during phase 3 (intraoperative), by simplifying the principle and by optimizing performance. Indeed, the use of holographic surgical navigation replaces the heavy and instrumented iterative operation of a passive assistance station by providing the surgeon, in his operative field of vision, with 3D scenes constructed during phase 2 of surgical planning superimposed on reality. The surgeon therefore uses his hands or even his voice to manipulate holograms in order to adapt the 3D scenes to his liking and guide himself using the information provided. One of the major advantages of this technology is harmlessness to the patient: apart from the visualization system that the practitioner must wear, no additional equipment is required, reducing the risk of contamination in the operating room. It is of course necessary to validate the compatibility of the ergonomics of the augmented reality visualization system with the use in question so as not to integrate any risk of hindering the surgical procedure due to the presence of the holograms.

The surgeon thus finds himself in a situation where he can integrate steps into his patient care process that create a direct link between the diagnosis and the guidance of his surgical act through surgical planning, using the set of patient data from imaging to complete the process. He can also choose, depending on his needs, to use the surgical planning to manufacture implantable medical devices or ancillary instrumentation to assist the surgical act specific to the patient's anatomy. The structuring nature of the process of phases 1 to 3 therefore implies strong interdependence. They must be the links in a chain that is as linear as possible in order to combine performance and safety, the two major requirements of medical device regulations.

### From Industry 4.0 to Surgery 4.0

This new approach to flow processes in structuring patient care is thus characterized by a holistic understanding of successive operational issues, integrating both technical and medical requirements, while seeking to take advantage of the most advanced computer technologies. The resulting concept is thus very similar to that of Industry 4.0, which consists of making a strong and innovative industry sustainable by bringing together the fields of virtual and reality around digital modeling.

Through ongoing and real-time communication between the elements and actors in the operational chain, this convergence thus allows strong customization at controlled costs, despite the low volumes produced. Industry 4.0, corresponding to the 4th industrial revolution after mechanization (1st), mass production (2nd) and automation (3rd), focuses on the generalization of so-called factories “of the future” that natively integrate modeling, personalized production and communication technologies for their operation: 3D simulation, cloud computing, artificial intelligence, additive manufacturing, virtual reality, augmented reality, etc. The transposition of these principles to the field of interest in the present invention in order to structure patient care from planning to guidance in the operating room, with or without including the manufacture of implantable or ancillary medical devices, gives life to a concept that the authors refer to as “Surgery 4.0.”

This concept can be generalized to any type of surgery using 3D imaging (generated directly or via the compilation of 2D elements). One of the most demanding medical specialties, among the most emblematic of the problem that the invention addresses, is orthopedic and traumatological surgery (which may be jointly referred to hereinafter using the single commonly accepted term “orthopedic surgery”), mentioned throughout this analysis of the prior art. This non-limiting example is sufficient to concretely illustrate the need to remedy all or part of the drawbacks of the prior art described above, as well as the solutions provided by the present disclosure. This introduction was written with respect to a human patient. The same drawbacks could be described with a patient of another animal species.

### The System

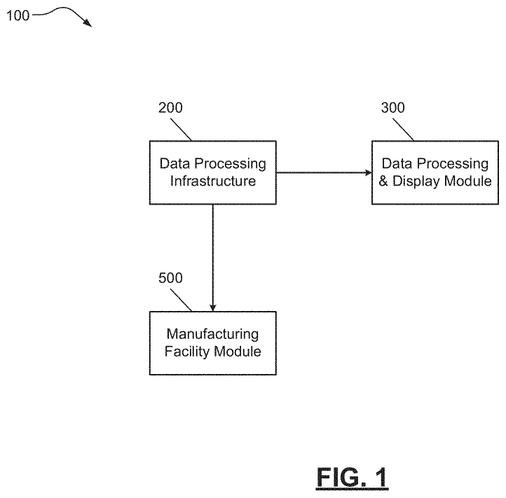

FIG. 1 illustrates a system 100 for assisting in setting up a personalized therapeutic approach for patients subject to medical and surgical care. The system 100 includes: (i) a data processing infrastructure 200 for data processing, (ii) a data processing and display module 300 for processing and displaying data, and (iii) a manufacturing facility module 500 for manufacturing medical devices of the implant or ancillary instrumentation type, whether standard or specific to the anatomy of the patient, or anatomical elements of said patient.

The data processing infrastructure 200 includes a processor and memory for data storage. The data processing infrastructure 200 may be connected via a distributed communications network, such as the Internet, in a cloud application platform allowing the exploitation and delivery of resources and services over the Internet by data storage means on a remote server via a cloud computing solution. In various implementations, the data processing infrastructure 200 may be implemented on a local data storage on individual computers.

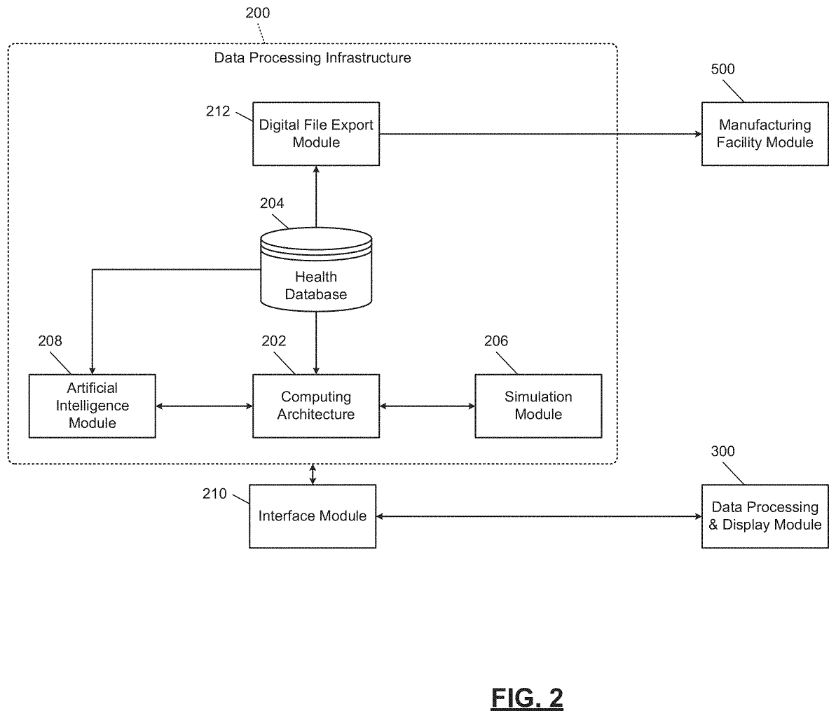

FIG. 2 is an example functional block diagram of the data processing infrastructure 200. The data processing infrastructure 200 includes (i) a computing architecture 202, (ii) a data storage device, including health database 204, (iii) a simulation module for physical systems simulation using numerical mathematic modeling 206, (iv) an artificial intelligence module 208 containing computer programs intended to implement algorithmic functions capable of simulating the human intelligence of the operator, (v) an interface module 210, accessible by third-party systems, and (vi) a digital file export module 212 for manufacturing devices, in particular medical, implantable or ancillary devices, which may or may not be specific to the patient's anatomy, or anatomical elements of said patient. In the remainder of the disclosure, non-specific devices are defined as “standard”.

In various implementations, a patient can be a human or other animal species. According to at least one embodiment, the data processing infrastructure 200 may exclude the simulation module 206 for physical systems simulation using numerical mathematic modeling. Additionally or alternatively, the data processing infrastructure 200 may exclude the artificial intelligence module 208.

The computing architecture 202 is of the type with shared resources, unified data and is accessible remotely by one or more simultaneous software clients. Sharing resources allows the solution to evolve in response to increasing use. Unifying the data makes it possible to secure the integrity thereof, preventing the data from being altered intentionally or unintentionally by fraudulent modification or degradation of the initial data through successive copying. The accessibility of the architecture is made possible by an open API (Application Programming Interface) of the network type, ensuring the correct distribution of the architecture.

The computing architecture 202 can, for example, include a farm of machines, each of the machines comprising a CPU (Central Processing Unit), and able to integrate a GPU (Graphical Processing Unit), intended to operate a cloud-based 3D rendering and computing farm (example: 3DVerse solution www.3dverse.com).

The GPUs deployed within the framework of the architecture 202 can, for example, include professional NVIDIA Ampere-type graphics processors with integrated graphics processing functionalities making it possible to provide 3D rendering in order to meet the requirements of massively parallelized computations.

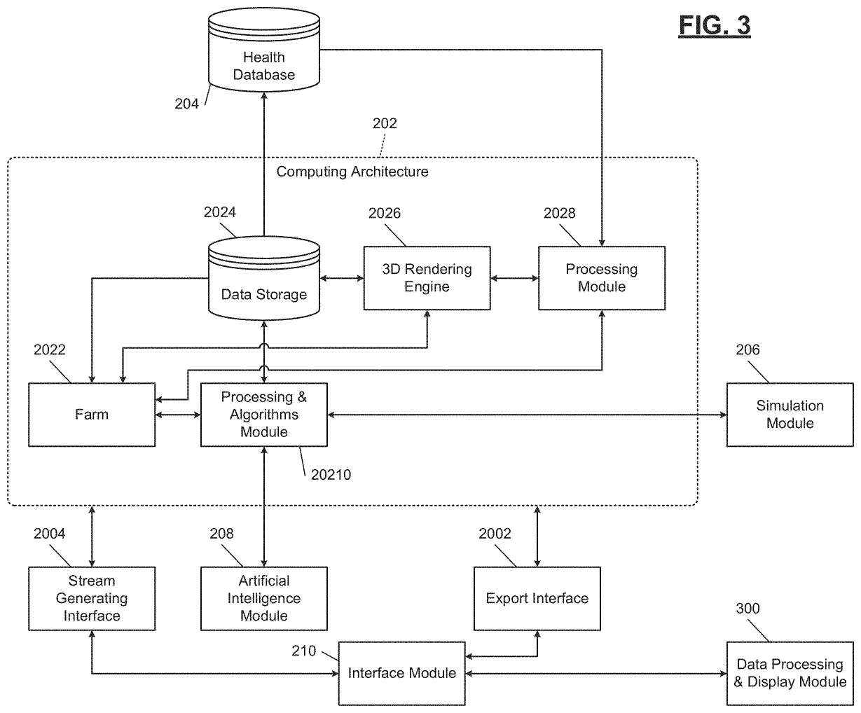

FIG. 3 is an example functional block diagram of the computing architecture 202. The computing architecture 202 more particularly includes (i) a farm 2022 of virtual or physical machines and (ii) a data storage device 2024. The computing architecture 202 aims to provide: a 3D rendering engine 2026, a processing module for three-dimensional reconstruction of computer-assisted imaging data 2028, and a module 20210 for processing data and algorithms.

The farm 2022 of virtual or physical machines can typically include virtual machines with a computing power of several teraflops intended to run the rendering engine 2026 and the processing and algorithms module 20210. The machines are shared between several operators, and their number can grow automatically based on the needs and the number of simultaneous operators. Typically, a machine can provide the services of the 3D rendering engines 2026 and the module 20210 for one or more operators and the assembly 2022 is scaled based on the number of simultaneous operators.

The storage device 2024 allows the storage and management of a variety of items, including: operator account management, 3D scenes of the computing architecture 202, binary files of the assets making up the 3D scene. These assets can be in particular 3D objects in voxels, in mesh, 2D or 3D textures or materials, the code files of the algorithms executed by the module 20210.

The 3D rendering engine 2026 can be implemented in the form of software or algorithmic functions integrated into special graphics cards (hardware type) that compute a 3D scene by restoring the 3D projection, the textures (appearance of the surfaces of the visualized objects), lighting effects (shadows, reflections, etc.), or even physical behaviors such as deformations of soft bodies, rigid bodies, particular behaviors or even the behaviors of fluids (liquids, gases, etc.). The whole constitutes a chaining of functionalities to form a coherent channel for the dissemination and successive processing of graphic information from the raw data to the operator terminal (Graphics Pipeline). The types of 3D rendering engines 2026 include, but are not limited to, engines with software acceleration and drivers with hardware acceleration.

Within the scope of the invention, it is preferable to implement motors with hardware acceleration using the computing power of the set of machines or farms 2022. The 3D rendering engine 2026 of the computation architecture 202 combines data of the voxel type and data of the mesh type displayed in a single 3D scene, while respecting their respective scales.

The 3D rendering engine 2026 is configured to generate a 3D scene from input data. The input data for the 3D engine can come from the storage device 2024. The input data of the 3D engine can come from data generated by the processing module 20210.

The 3D scene whose objects are contained in the storage device 2024 can be exported via an export interface 2002 of the computing architecture 202, which can be accessed through the module 210. The 3D scene generated by the 3D rendering engine 2026 can be broadcast in streaming form to one or more instances of the module 300, via a stream generating interface 2004.

The computer-assisted imaging data three-dimensional reconstruction processing module 2028 has an input interface for receiving data, as well as an output interface for transmitting processed data. The input data is received from the storage device or health database 204, which in turn is fed by the files coming from medical imaging examinations and downloaded to a remote server by the operator of the application. The input data are data from volume imaging examinations with 3D reconstruction via back projection algorithms, for example of a DICOM-type tomodensitometric nature (X-ray tomography or MRI, for example).

The output data is voxel-type data (3D texture). The processing module is also capable of transforming voxel-type data into mesh-type data representing the outer part of the object as a voxel. The processing includes computing a three-dimensional scene corresponding to an area of a body, human or animal, for example from DICOM-type data. The voxel- and mesh-type objects obtained in the form of binary files as output of the processing module as well as the scene composition information are hosted within the storage device 2024.

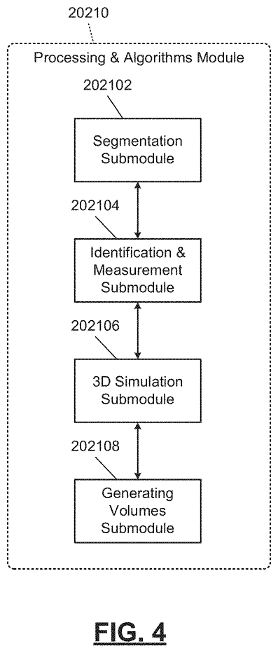

FIG. 4 is an example function block diagram of the processing and algorithms module 20210. The data processing module or processing and algorithms module 20210 is configured to receive input data, process the input data, and generate output data. The processed data is stored within the storage device 2024.

The data processing module 20210 includes a submodule 202102 for segmentation of the biological elements of interest, a submodule 202104 for identification and measurement tools to assist in the characterization of the patient's pathology, a submodule 202106 for 3D simulation of a surgical sequence as a unitary element of the surgical treatment, and a submodule 202108 for generating volumes corresponding to implantable medical devices and/or ancillary surgical assistance instrumentation, which can both be standard or specific to the patient's anatomy, or anatomical elements, from output data of the submodule 202106.

To perform the various processing operations, the processing and algorithms module 20210 uses the computing power of the farm 2022. The output data can be addressed to the storage device 2024.

The submodule 202102 for segmenting biological elements of interest from the medical imaging data is configured to determine a segmentation from the input data. These input data constitute, after processing of the initial imaging examination of the patient in DICOM format by the 3D reconstruction module 2028, a 3D object preferably in voxels stored in the database 2024 for keeping a high level of fidelity to the patient's anatomy.

The submodule 202102 performs the following operations: generating several 3D textures representing the different types of elements of interest, for example different types of tissues (bones, muscles, air, etc.) depending on the intensity level of each voxel, and extracting individual anatomical elements, for example specific bones (femur, tibia, radius, vertebrae, jaws, etc.) as part of an application to orthopedic or maxillofacial surgery, by different possible methods. A non-exhaustive list of different methods includes: clipping by operator selection of voxels with a manual tracing tool, selection of contiguous voxels, propagation/expansion of the selection, identification of concave shapes to delimit the articular surfaces, shape recognition, co-location of the anatomical parts by statistics in order to predict whether a voxel belongs to an organic tissue. Optionally, the method may include processing and refining of the segmentation with smoothing or correction operations of the 3D structure.

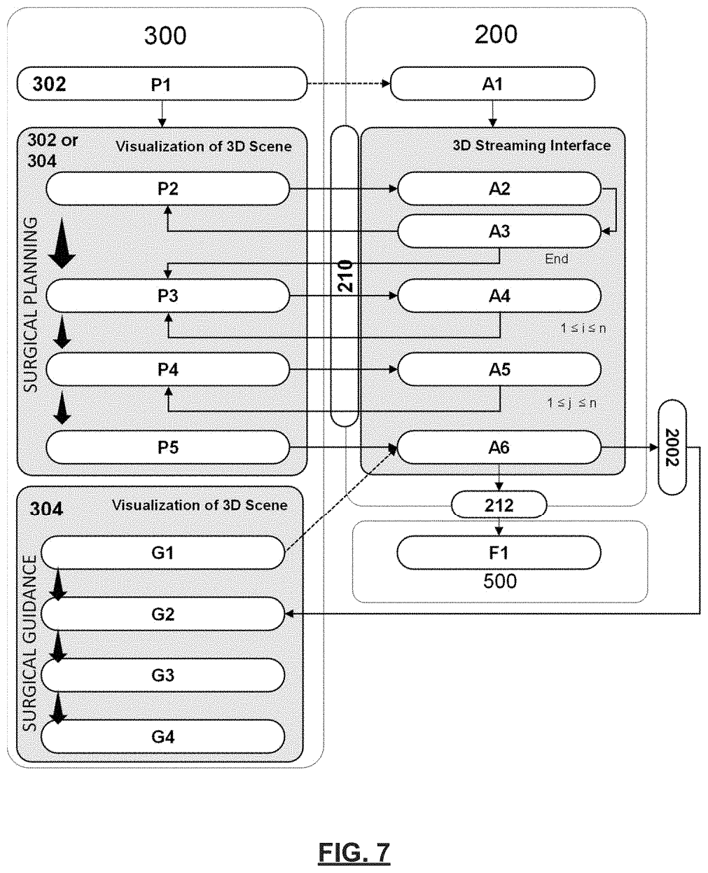

For example, segmentation can relate to tissues of any kind (bone, tendon, epidermal, dental, vascular tissues, etc.), anatomical elements such as individual bones (femur, tibia, etc.) or even an organ such as the liver, the pancreas, the prostate or the heart depending on the medical specialty in question. The segmentation submodule 202102 is used in step A2 of the flowchart shown in FIG. 7.

The identification and measurement submodule 202104 is configured to assist the operator with a view to characterizing the patient's pathology, for example from input data or from input data segmented by the data segmentation submodule 202102. This characterization is carried out from the observation of the original 3D object in voxels and/or from the segmented elements originating from the submodule 202102, stored in the storage device 2024. The operator can nevertheless choose to work on non-segmented input data resulting from the processing of the module 2028 and hosted in the storage device 2024. The operator can also add input data at this stage to structure the rest of the process by entering additional information in the system. This can, for example, constitute annotating the 3D anatomical scene (additional pathology identification, identification of anatomical landmarks, etc.). The term “labeling” is also often used to describe the annotation action, in particular for the purpose of training artificial intelligence algorithms.

According to one particularity, in application to orthopedic surgery, the identification and measurement sub-module 202104, for example, provides the information necessary for the parameterization of a surgical treatment of bone deformation by corrective osteotomy, including: identifying the presence or absence of a bone deformity by comparing, if applicable, bones of the same type on the patient's right and left side. This is the case, for example, with so-called “long” bones of the appendicular system in mammals such as humans or dogs, for example: tibia, femur, humerus, radius, ulna, and, for the same type of bone, bones of the same type compared to healthy, undistorted bone models.

The identification and measurement sub-module 202104 further provides the necessary information for measuring the length of pathological and healthy bone (if applicable), identifying and measuring the anatomical axis of the pathological bone, identifying and measuring the mechanical axis of the corrected bone, measuring the articular angle between the corrected bone and the bone(s) attached to said joint, annotating anatomical landmarks on the pathological bone, and annotating required information (possibly three-dimensional diagrams) for the continuation of the planning process. The identification and measurement submodule 202104 is, in particular, used in stage A3 of the flowcharts shown in FIGS. 7-9.

The submodule 202106 for simulation of a surgical sequence S(i) is configured to compute a 3D sequence corresponding to a stage of the surgical procedure envisaged by the operator; this sequence is also identified by the expression “operating time.” The term surgical sequence is used in the remainder of the document. The surgical treatment modeling submodule 202106 uses the input data from the module 2028 or the input data segmented by the data segmentation submodule 202102, or the patient identification and measurement data generated by submodule 202104. The surgical sequence simulation submodule 202106 is configured to perform the following operations, from the segmented elements of interest from the submodule 202102 and 202104, elements stored in the storage device 2024: (1) allowing the operator to manipulate the different elements from the submodules 202102 and 202104 and (2) proposing an assistance sequence S(i) for modeling of the surgical treatment planned for the operation. This sequence must allow the operator to validate or adjust the parameterization of the modeling of said operation.

The simulation of a step, automated or not, is a direct function of the planned medical procedure, each procedure being able to be broken down natively into distinct sub-phases from one medical specialty to another. Each sub-phase corresponds to a step S(i), the maximum value of the variable “i” corresponding to the total number of sequences to be simulated (by convention, we will write 1<i≤n).

Thus, according to a first feature, applied to orthopedic surgery for the treatment of correction of a bone deformation by osteotomy, this could correspond to the following series of sequences for n=4. S(1) or the first sequence includes computation and display of a 3D scene showing the computations (equation, positioning, etc.) and positioning on a deformed bone of one or more bone correction cutting plane(s).

S(2) or the second sequence includes computation and display of the result of the application of the bone section displayed in S(1) on a deformed pathological bone to allow the operator to see the visual result of the virtual deformation correction act. S(3) or the third sequence includes display of a 3D scene showing the level of contact surface between the different bone segments of interest resulting from the section of the pathological bone as a function of the manipulation of these bone segments. This step makes it possible to virtually model the surgical act of adjusting the bone correction to be carried out in the operating room S(4) or the fourth sequence includes visualization of the bone after correction performed in S(3) upstream of the modeling of implantable medical devices and ancillary instrumentation for the osteosynthesis phase that will allow the correction to be fixed over time,

For all of the scenes S(i), the operator interaction is dynamic and makes it possible to observe the consequences in real time of a modification of the input data of the computation on the displayed result.

According to a second feature, applied to vascular surgery, this could correspond to the stages constituting the treatment of a venous thrombosis (obstruction of a venous duct by the appearance of a blood clot called thrombus) within a vascular system reconstructed in 3D. This treatment, called thrombolysis, can for example be simulated by a sequence S(1) incision, S(2) mechanical disaggregation, S(3) aspiration by catheter of the thrombus, S(4) closure (n=4).

According to a third possibility, applied to hepatic surgery, this could be the modeling of the treatment of resection of liver tumors after characterization of the tumor (dimensions, anatomical location) and simulate the successive surgical procedures S(i) of resection by laparoscopic instrumentation (medical endoscopy technique for intervention in the abdominal cavity—also called laparoscopy). The submodule 202106 for simulating a surgical sequence S(i) is used in step A4 of the flowchart shown in FIG. 7.

The submodule 202108 for digital generation of medical device DM(j) either standard or specific to the patient's anatomy is configured to transform the output data from submodule 202104 and/or submodule 202106. This submodule allows the operator to generate a number “j” of implantable devices and/or ancillary surgical assistance instruments in the form of 3D objects added to the main scene S(n). It is therefore a question of generating a three-dimensional representation of a standard medical device or one that is specific to the patient's anatomy morphology, the shape of which may depend on the characteristics of the anatomical area of interest defined by the operator by reproduction with a high level of precision of its topography. Such reproduction may include generating geometric volumes of any kind, whether unique or by assembling these volumes, which can be of standard shapes with axes of symmetry (cube, sphere, cylinder, etc.) or specific and generated according to the output data from the submodules 202104 and 202106. According to one particular feature, the generated implantable devices are osteosynthesis plates adapted to the morphology of a pathological bone (fractured bone, bone deformed then corrected), or else vascular stents with a design adapted to the vascular anatomy to be treated following thrombolysis, for example, generating a recess of suitable nature and geometry, which may or may not be a through recess, intended for example to receive medical devices generated by the same submodule or resulting from the operator's choice from a library of precomputed shapes, hosted from the storage device 2024 in anticipation of a permanent combination with the initial medical device at the time of surgical implantation.

This can also make it possible to optimize the 3D shape for the purpose of reducing the contact surface between the modeled medical device and the reference anatomical element. In the context of orthopedic and traumatological surgery, this can for example be screw-type bone anchoring systems chosen from among the component elements of a digital library, according to their type and dimensions, to be included within a bone plate for osteosynthesis or even temporary fixation devices of the bone fixation surgical pin type. According to one particular feature, the ancillaries for surgical assistance may be designated by the generic term “surgical guide” and possibly be composed of one or more elements for example integrating the functions of positioning and/or orientation of implantable device(s), cutting of biological tissue(s), or drilling bone, depending on the type of surgery (orthopedic, trauma, vascular, cardiac, neurosurgery, etc.).

According to a particularity in orthopedic and trauma surgery, it can be a custom-made cutting guide whose shape follows the surface of the bone at one or more location(s) defined by the operator by identically reproducing its topography, in particular including: one or more bearing surfaces for guiding a surgical cutting tool (example: oscillating saw) and/or a possibility of holes for temporary fixation devices of the surgical pin type.

In various implementations, it can be a custom-made drilling guide whose shape follows the surface of the bone at one or more location(s) defined by the operator by identically reproducing its topography, in particular including: a possibility of holes for guiding the drillings of holes dedicated to receiving bone anchoring systems of the screw-in-bone type and a possibility of holes for temporary fixation devices of the surgical pin type.

In various implementations, it can include generating a tailor-made orientation guide whose shape follows the surface of the corrected bone at the location defined by the operator by identical reproduction of its topography, in particular including: several holes for temporary fixation devices of the surgical pin type and a possibility of counterform to guide the positioning of the customized bone plate. The submodule 202108 for digital generation of medical device DM(j) either standard or specific to the patient's anatomy is used in step A5 of the flowchart shown in FIG. 7.

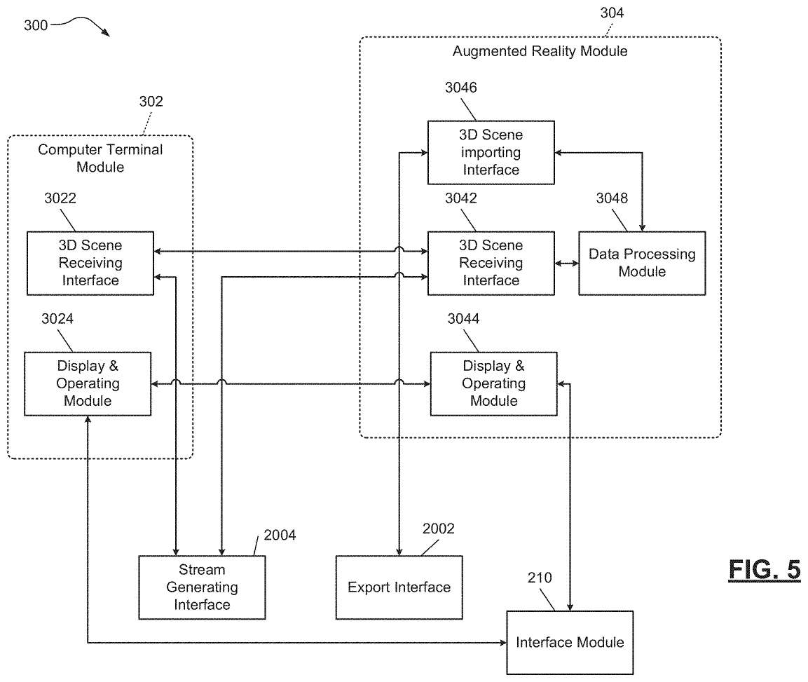

The health database storage device 204 in particular allows the management of operator accounts to connect to the data processing and display module 300, shown in FIG. 1. The data processing and display module 300 also includes medical data associated with patients, derived from medical imaging data, metadata associated with the medical imaging data and information entered by the surgeon operator of the postoperative assessment type, through the modules 302 and 304, shown in FIG. 5.

It also includes computerized 3D reconstructed medical imaging data associated with patients and postoperative assessment data entered in step G4. It also includes non-medical data, in particular for the management of operators, patient files and documents (orders, invoices, etc.).

The module for physical systems simulation using numerical mathematic modeling 206 uses, as input, the output data of the medical treatment modeling from the module for simulating a surgical sequence S(i) 202106 or module for generating a device volume 202108. It executes an algorithm making it possible to simulate and visualize the stresses and deformations induced within the assembly of the elements due to the mechanical stresses induced by the physical activity of the patient. The module uses the finite element analysis method to simulate the behavior of the assembly and identify the most stressed areas.

The submodule 202106 for simulating a surgical sequence S(i) can work in combination with the module 206 for physical systems simulation using numerical mathematic modeling in order to use the physical integrity of the anatomical part of interest as input data. This combination makes it possible to map the areas of fragility of the anatomical part of interest and to provide the operator with additional information to be taken into consideration when planning his surgery. When applied to tumor resection surgery, regardless of the concerned organ or tissue, this can thus provide information on the impact of the resection procedure on the tissues surrounding the ablation area by simulating the position of fragile areas when the standard stressing of the concerned anatomical portion is modeled. The submodule 202106 for simulating a surgical sequence S(i) combined with the module for physical systems simulation using numerical mathematic modeling 206 is used in step A4 of the flowchart shown in FIG. 7.

The submodule 202108 for simulating a surgical sequence S(i) can work in combination with the module 206 for physical systems simulation using numerical mathematic modeling in order to use the physical integrity of the anatomical part of interest as input data. This combination makes it possible to map the areas of fragility of the anatomical part of interest and to provide the operator with additional information to be taken into consideration when planning his surgery. When applied to trauma surgery for the treatment of a fracture, this can thus make it possible to model the implantable bone plate-type osteosynthesis device by taking into account the quality of the pathological recipient bone. Multi-fragmented bone requires special attention when positioning screw-type bone anchoring systems in solid areas. As a corollary, it is therefore necessary to model a plate which is both specific to this positioning of the screws and which, once associated with the recipient bone, makes it possible to maximize the long-term stability of the assembly {bone, bone anchoring systems of the screw, plate type}. The modeling of the constraints corresponding to a normal use of the limb by a breast patient (walking for the lower limb, for example) makes it possible to evaluate this stability over time and to validate—iteratively if necessary—the design of standard medical devices or those specific to the patient's anatomy. The submodule 202108 for digitally generating a standard or specific medical device DM(j) for the patient's anatomy combined with the module for physical systems simulation using numerical mathematic modeling 206 is used in step A5 of the flowchart shown in FIG. 7.

The artificial intelligence module 208 takes data from storage devices 204 and 2024 as input. It includes an annotation tool to categorize and label the information contained in the storage devices 204 and 2024, typically to classify the unitary elements making up the data sets used by the artificial intelligence algorithms. In addition, machine learning and deep learning algorithms can be called via interfaces that can be of the API type by the submodules 202102, 202104, 202106 and 202108 to simulate, in an automatic mode, the complex tasks that are due in manual or semi-manual mode to the operator, therefore the surgeon. The module 208 thus makes it possible to categorize the actions of these submodules in a “smart” mode, to be broken down into sub-tasks or a set of automatic sub-tasks that do not require operator intervention to characterize an input datum and transform it into output data. These tasks include automatic segmentation based on the submodule 202102 of the different elements such as for example the different types of tissues (epidermal, vascular, bone, etc.) or the bones of the skeleton (femur, tibia, vertebra, pelvis, etc.) (step A2 in FIG. 7).

The tasks also include identification and automatic measurement based on the submodule 202104, to carry out analyses of the 3D model allowing the operator to accurately characterize the patient's pathology with a view to the surgical procedure in an automated or semi-automated mode by partial automated assistance during the process. In one possibility of the invention, this module provides the necessary information to the operator, who will then be able, based on his training and his experience in surgery, to strengthen his diagnostic thinking in order to continue the surgical planning process. In an alternative possibility, the module 208 directly provides the operator with a characterization of the pathology that the operator will need to confirm or reject in order to move on to the next stages of surgical planning (step A3 of FIG. 7).

The tasks further include automatic 3D simulation of a surgical sequence S(i) of the operation based on the submodule 202106 from the output data of the submodule 202104. This simulation allows the operator to perform an automated or semi-automated simulation by partial automated assistance during the process. In one possibility of the invention, this module allows the operator to predefine selection criteria, such as, for example, the preservation of the anatomical and mechanical axes of a fractured bone with a view to simulating the surgical sequence of osteosynthesis within the orthopedic specialty (step A4 in FIG. 7).

The tasks additionally include automatic digital generation of standard medical device or device specific to patient anatomy DM(j) based on the submodule 202108, from the output data of the submodule 202106. The association with the module 208 thus makes it possible to generate and position, in an automatic or semi-automatic mode (depending on the degree of assistance), implantable medical devices or ancillary instrumentation for assistance with surgery, for example osteosynthesis systems {bone plate, bone anchoring systems} in order to obtain the best stability of the assembly in the context of fracture repair, as well as all of the associated surgical guides. It can also be to automatically simulate a vascular stent (step A5 of FIG. 7).

In accordance with the content of the flowcharts of FIGS. 7-9, the combination of one or more of these smart versions of the submodules 202102, 202104, 202106 and 202108 allows all or part of the surgical planning to be automated: including: characterization of the pathology, modeling of the surgical act, and modeling of a standard medical device or a medical device specific to the patient's anatomy.

The interface module 210 is made up of a set of functions allowing and facilitating communication between the data processing infrastructure 200 and third-party applications with the objective of exchanging services or data with one another. These interfaces can be, but are not limited to, API, web service or file exchange type interfaces.

The digital file export module 212 exports files for manufacturing medical devices of the implant or ancillary instrumentation type, whether standard or specific to the anatomy of the patient, or anatomical elements of said patient. The digital file export module 212 for the manufacture of implant-type medical devices, or ancillary surgical assistance instrumentation, whether standard or specific to the patient's anatomy 212, or anatomical elements, takes data from storage devices 204 and 2024 as input, from stages A1, A2, A3, A4 and A5. It executes a set of algorithms that generate files intended for the manufacturing facility module 500, which may contain in particular: digital files, a metadata file, and/or 2D representations.

The digital files can represent implantable devices modeled from the patient's anatomy (e.g. bone plate in orthopedic surgery); ancillary surgical assistance instruments also modeled after the patient's anatomy, possibly of the “guide” type, tailored; or even anatomical elements. These files are typically in a format which can in particular be STL (abbreviation for Stereolithography), AMF (Additive Manufacturing File format) or even OBJ (Object file).

The metadata file can include additional information necessary for manufacturing (dimensional marks, specific geometric information, control beacons, positions and typology of specific locations intended to receive durable or temporary implantation systems for surgical needs). The 2D representations make it possible to generate industrial-type definition plans, which can be used directly for manufacturing. The digital file export module 212 is used in step A6 of the flowchart shown in FIG. 7.

FIG. 5 is an example functional block diagram of the data processing and display module 300. The data processing and display module 300 is for processing and displaying data intended for operators. The data processing and display module includes a computer terminal module 302 for processing and displaying data intended for any type of computer terminal. A non-exhaustive list includes: computer, smartphone, tablet, virtual reality device, augmented reality device, etc. The data processing and display module 300 also includes an augmented reality module 304 for processing and display intended for any type of computer terminal allowing visualization in augmented reality.

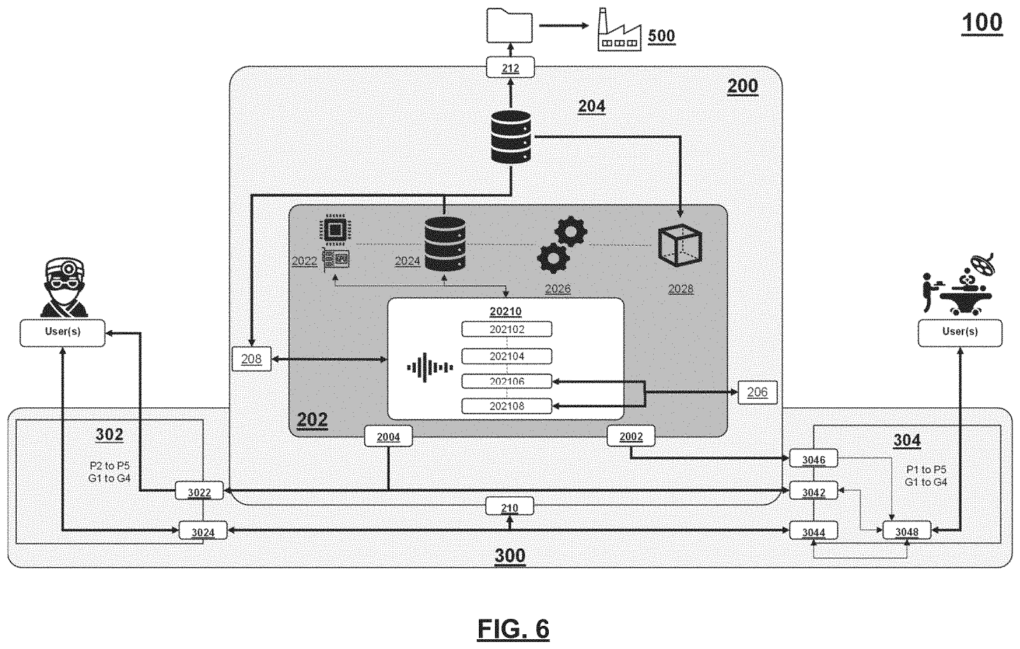

FIG. 6 is a functional block diagram depicting the connections between the various modules of the system. Several simultaneous executions of the computing terminal module 302 or the augmented reality module 304 can connect together with the data processing infrastructure 200 or module and visualize and manipulate the same information and objects. This possibility offered by the system that is the subject of this document thus allows remote or local collaboration between several operators, possibly to share the experience of surgical planning and surgical guidance, but also for assistance between peers, professionals in the medical field, for the purpose of assistance in the assessment of pathological cases or medical training.

The computer terminal module 302 is in the form of an application of the heavy client (local) type installed on the operating system or of the light client (web) type accessible through an Internet browser. This computer terminal module 302 is for example accessible from a computer terminal (computer, smartphone, tablet, virtual reality display device, augmented reality display device, etc.). This computer terminal module 302 in particular includes: a 3D scene receiving interface in the form of a stream 3022 and a display and operating module 3024 making it possible to interact with 200 through the interface module 210.

The interface module 210 allows the operator to carry out surgical planning, whatever the medical specialty, via a series of steps, shown in the flowchart of FIG. 7. Step P1 includes uploading of medical imaging files, for example of the DICOM type, to the storage device, which may include health database 204 via the interface 210. Step p1 may be performed by the computer terminal module 302. The data is then processed by the module 2028 in order to be generated in three dimensions and constitute the initial 3D scene of the whole process visualized in the form of a 3D scene by the module 202 and received through the stream generating interface 2004.

All of the steps of P2 to P5 that make up the rest of the process allow the manipulation and creation of 3D objects of the 3D scene with the operator interface devices of the computer terminal used (computer, smartphone, tablet, etc.), by relying on the interfaces 210 provided by the infrastructure 200. Steps P2 to P5 may be performed by the computer terminal module 302 or the augmented reality module 304.

Step P2 includes characterization of the pathology affecting the patient by successive and possibly iterative execution of the following two submodules: tissue segmentation by calling the submodule 202102 through interfaces 210 provided by the infrastructure 200 and identification and measurement by calling the submodule 202104 through the interfaces 210 provided by the infrastructure 200.

Step P3 includes modeling of surgical procedures by “i” calls (1≤i<n) of the 3D simulation submodule of a surgical sequence S(i) 202106 through the interfaces 210 provided by the infrastructure 200. Like for step P2, several successive calls can thus be made to simulate the number of surgical sequences required by the planning of the pathology of interest. Each call generates a different 3D scene from the previous one (n scenes).

Step P4 (if applicable) includes modeling of a standard medical device or a medical device specific to the patient's anatomy by “j” calls (1≤j≤m), m corresponding to the number of modeled devices) of the submodule for digitally generating a standard medical device or a medical device specific to the patient's anatomy DM(j) 202108 through interfaces 210 provided by the infrastructure 200. Like for the steps P2 and P3, several successive calls can thus be made to simulate the number of medical devices required by modeled medical treatment. Step P5 includes review and validation of the planning by visualizing the different steps P2 to P4 (or P3).

The interface also makes it possible to enter the information from the postoperative assessment in step G4 for the surgical guidance (FIG. 7), which may in particular include dictated or typed text transmitted to the health database storage device 204 through the interface 210 or medical imaging files possibly of the DICOM type transmitted to the module 2028 through the interface 210.

According to one possibility of the present disclosure, step P2 can integrate a function for exporting the elements displayed or chosen by the operator in digital format for production by the manufacturing facility module 500 via the module 2002, and possibly those stated in the introductory part. According to one possibility of the invention, step P4 is optional, and the planning of the surgical procedure may be sufficient in order to then carry out the surgical guidance.

Returning to FIG. 5, the augmented reality module 304 is in the form of a software application embedded in augmented reality systems. The augmented reality module 304 includes an interface for receiving a 3D scene in streaming form 3042 and a display and operating module 3044 making it possible to interact with 200 through the interface 210. The augmented reality module 304 includes an interface for importing a 3D scene 3046, connected to the interface 2002 for exporting the computing architecture 202, potentially through the interface 210, used in step G2 and a data processing module or unit 3048 arranged to receive data from the 3D scene import interface 3046 and data from the 3D scene import interface in streaming form 3044.

The data processing unit 3048 is configured to generate a user interface for the operator for display by the holographic display terminal. This unit also processes information from sensors that can for example be depth cameras, infrared sensors or transmitters in order to determine the correspondences between spatial reference frames and to allow the projection of the holograms on the anatomical reference areas of the patient to guide the operator as well as possible, and in particular the surgeon (for example: projection of information, measurements, 3D objects modeled during planning, etc.).

The interface allows the operator to perform surgical planning in a manner analogous to what has been described previously, in particular via the following operations in holographic visualization for steps P2, P3, P4, and P5. The holographic visualization includes visualization of 3D scenes generated by the module 202 and received through the stream receiving interface 3042 and the stream generating interface 2004, or by importing a 3D scene generated by the module 202 and received through the 3D scene import interface as a stream 3046 and the export interface 2002. The holographic visualization includes manipulation of 3D objects from the 3D scene with hands or dedicated devices, relying on the data processing unit 3048 and the interfaces 210 provided by the infrastructure 200.

Then, more specifically for each stage of surgical planning, step P2 includes characterization of the pathology affecting the patient by successive and possibly iterative execution of the following two submodules: tissue segmentation by calling the submodule 202102 through interfaces 210 provided by the infrastructure 200 and identification and measurement by calling the submodule 202104 through the interfaces 210 provided by the infrastructure 200.

Step P3 includes modeling of surgical procedures by “i” calls (1≤i<n) of the 3D simulation submodule of a surgical sequence S(i) 202106 through the interfaces 210 provided by the infrastructure 200. Like for step P2, several successive calls can thus be made to simulate the number of surgical sequences required by the planning of the pathology of interest. Each call generates a different 3D scene from the previous one (n scenes).

Step P4 (if applicable) includes modeling of a standard medical device or a medical device specific to the patient's anatomy by “j” calls (1≤j<m≤n) of the digital generating submodule of a standard medical device or a medical device specific to the patient's anatomy DM(j) 202108 through interfaces 210 provided by the infrastructure 200. Like for the steps P2 and P3, several successive calls can thus be made to simulate the number of medical devices required by modeled medical treatment. Step P5 includes review and validation of the planning by visualizing the different stages P2 to P4 (or P3). According to one possibility of the invention, step P4 is not required, and the planning of the surgical procedure may be sufficient in order to then carry out the surgical guidance.

The augmented reality module 304 is also configured to provide the surgeon with a real-time visualization tool, possibly manual or in an automated mode, embedded in a device that is preferably individual as described above, compatible with use in the operating room during a surgical procedure, for holographic guidance purposes to accompany the operator practitioner during the performance of the operation planned in advance and validated in step P5. This surgical guidance tool takes the form of a user interface that enables the steps G1 through G4 to be performed, as shown in FIG. 7.

Step G1 includes initiation of the surgical guidance process by selecting an operation planned in advance during this first step, possibly presented as a file relating to said operation, referring explicitly or anonymously to the patient concerned by the operation, and/or to the expected date of surgery. The module 304 then connects to the 3D rendering engine 2026 via the stream receiving interface 3042 to display the objects resulting from steps P1 to P5, possibly in a sequential mode, or optionally by importing the data from the export interface 2002 during this step (step A6 of FIG. 7).

Step G2 includes visualize the surgical planning carried out beforehand and validated in step P5, typically in the form of holograms including in particular: 2D or 3D representation of the medical imaging examination, possibly in a combined presentation of both types of formats, allowing simultaneous navigation in these multiple representations based on the operator's input, for example according to the multiplanar reconstruction model used in medical imaging for the analysis of files (for example CT scans). The holograms further include the 3D scenes from the pathology characterization step as output data from the submodules 202102 for tissue segmentation and 202104 identification and measurement. The holograms also include 3D scenes from the surgical act modeling step as output data from the submodule 202106 for 3D simulation of a surgical sequence S(i). This possibly includes the 3D view of the anatomical element of interest before and after surgical treatment if applicable (for example: bones before and after reconstruction as part of the treatment of a fracture in orthopedic surgery) as well as the information and notes entered or computed during planning.

The holograms additionally include, if applicable, the 3D scenes from the stage for modeling standard medical devices or medical devices specific to the patient's anatomy as output data from the submodule 202108 for digital generation of a standard medical device or a medical device specific to the anatomy of the patient DM(j). In the context of fracture repair in trauma surgery, this for example corresponds to bone anchoring systems of the screw type, temporary fixation devices of the surgical pin type (example: Kirschner wire), a bone plate or even surgical guides.

The holograms further include manipulating 3D objects with the hands or with the help of dedicated devices, by relying on interfaces 210 provided by the infrastructure 200 and performing measurement operations, for example distance and angles on holograms or on the patient.

Step G3 includes functionality of automatic positioning on the patient during the intervention of the elements resulting from the surgical planning and shown in 3D in holographic form. In the context of fracture repair in trauma surgery, this for example corresponds to the automatic positioning of standard medical devices or medical devices specific to the patient's anatomy: bone anchoring systems of the screw type, temporary fixation devices of the surgical pin type (example: Kirschner wire), osteosynthesis plate or even surgical cutting, orientation or drilling guides for example. This so-called “recalibrated” positioning of holograms on the patient can be done for example by algorithms of the artificial intelligence module 208. These algorithms perform a real-time comparison of the anatomical data of the patient from the original medical imaging examinations used for the surgical planning and the information from sensors that can for example be the video systems coming from the augmented reality display device during of the surgical procedure. These algorithms could for example operate on the principle of computer vision to determine the correspondences between spatial reference frames and allow the projection of the holograms on the anatomical reference zones of the patient in order to best guide the surgeon (for example: projection of information, measurements, 3D objects modeled during planning, etc.).

Step G4 includes entering the information from the operating report, which may in particular contain: captures in the form of images or video sequences done during the surgical operation by the augmented reality visualization system. These captures are obtained either by triggering by the operator (for example: by voice or by gestures) or automated by a prior parameterization carried out by the operator during the surgical planning. These captures are transmitted to the storage device for health database 204 through the interface 210 and/or text dictated or typed using a virtual keyboard transmitted to the health database storage device 204 through the interface 210.

Facility (500) is for manufacturing medical devices such as implants or ancillary instrumentations, standard or specific to the anatomy of the patient, or anatomical elements of said patient. In the embodiment described above, the system 100 includes installation of the manufacturing facility module 500. According to a variant of the invention, the system 100 does excludes the manufacturing facility module 500.

The manufacturing facility module 500 allows the use of files in digital format from the export module 212 for the manufacture on demand of medical devices of the implant type or ancillary instrumentation the anatomy of the patient, or of anatomical elements of said patient. The manufacturing facility module 500 can be a subtractive (for example machining) or additive (for example 3D printing) manufacturing method allowing the shaping of standard medical devices or medical devices specific to the patient's anatomy. The materials used, which are compatible with health use, can be metallic, ceramic, plastic, organic or composite.

The manufacturing facility module 500 also integrates all the post-treatment steps of standard medical devices or those specific to the patient's anatomy to allow their future use in surgery. These steps are typically as follows (but are not limited to): one or more heat treatment step(s), one or more surface treatment step(s), one or more medical grade cleaning step(s), one or more conditioning step(s) in environments with controlled or uncontrolled atmospheres, and a step for sterilization of the finished products.

The facility also makes it possible, where appropriate, to manufacture anatomical elements such as organs or portions of organs, or even bone elements (fragments, individualized bones, etc.).

### Use Cases of an Architecture According to the Invention

The architecture according to the invention is implemented by a surgeon during the three stages of planning, manufacturing, and guiding. During the manufacturing step, one or more anatomical elements, one or more implantable medical devices and/or one or more ancillary instrumentation medical devices can be manufactured. The architecture according to the invention can also be implemented in collaborative form during one or more of the various aforementioned steps.

According to a first possibility, the architecture according to the invention can be implemented for surgery with remote preoperative assistance for which the surgeon requests one or more third person(s) for assistance in preparing for the surgery, during the planning stage, remotely, for example one of his colleagues of the same specialty, a colleague from another specialty, for example a radiologist. The surgeon can also seek the advice of other technical experts (support in the use of new surgical equipment for example). All of the third-party consultations can also be combined in order to help the surgeon using the solution adopt the best therapeutic approach. In some health systems such as the French system, the act of requesting third-party assistance in medical imaging to confirm or refute a diagnosis is called an act of “remote assistance”. This action is frequent in more traditional ways among radiologists or between surgeons and radiologists.

According to a second possibility, the architecture according to the invention can be implemented for surgery with remote intraoperative assistance for which the surgeon requests one or more third parties for remote surgical assistance during the procedure, for example one of his colleagues in the same specialty, or a colleague from another specialty, for example a radiologist. The surgeon can also seek the advice of other technical experts (support in the use of surgical equipment for example).

According to a third possibility, the preoperative assistance described according to the first possibility and the intraoperative assistance described according to the second possibility can be combined.

According to a fourth possibility, the architecture according to the invention is implemented by the surgeon only during a planning step, with a manufacturing step reduced to the manufacture of an anatomical element, and without an intraoperative guidance step. This usage scenario allows the surgeon to explain, with the visual aid formed by the anatomical element, the consequences he envisages for the patient. The result may be not to operate.

Each of the preceding possibilities, when it provides for the implementation of a manufacturing step, generates a new usage scenario, in which the manufacturing step is removed from said possibility.

Of course, the invention is not limited to the examples which have just been described, and numerous modifications can be made to these examples without departing from the scope of the invention. In addition, the various features, forms, variants and embodiments of the invention can be associated with each other in various combinations as long as they are not incompatible or mutually exclusive.

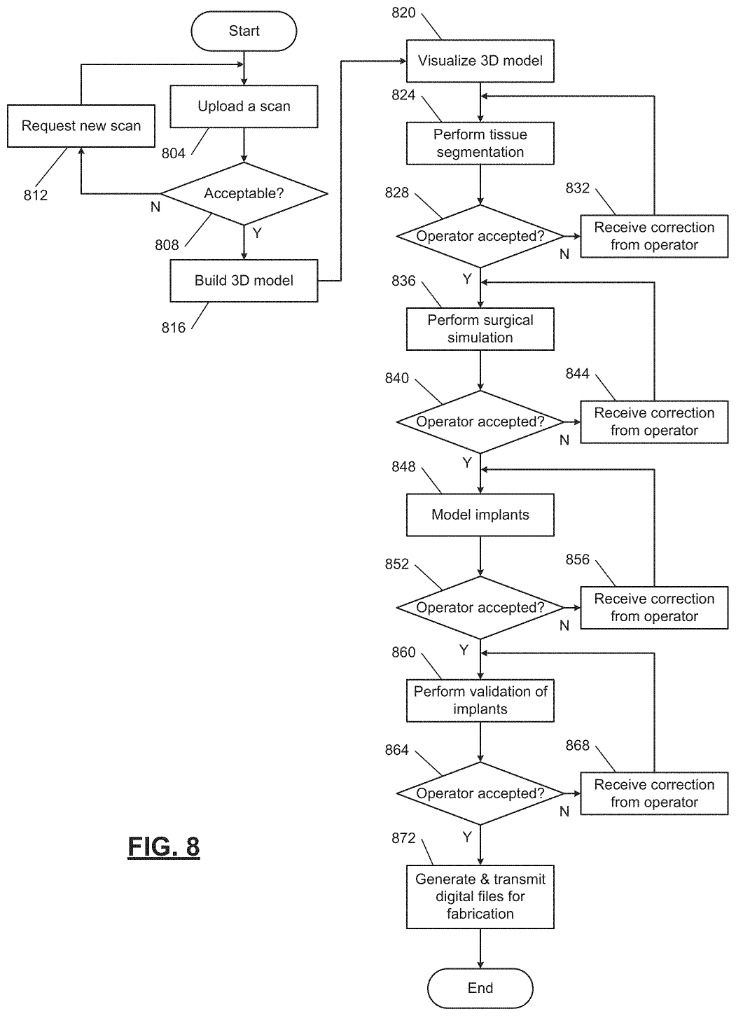

FIG. 8 is another example flowchart describing the specific implementation steps of surgical planning (P2 to P4). Control begins when a scan is uploaded at 804, for example, a CT scan or other medical imaging file. Control determines, at 808, if the scan is acceptable. If no, control proceeds to 812 to request a new scan. Control then returns to 804. Otherwise, if the scan is acceptable, control continues to 816 to build a three-dimensional model based on the uploaded scan. Control continues to 820 to visualize the three-dimensional model. At 824, control performs tissue segmentation on the three-dimensional model. At 828, control determines if the operator has accepted the tissue segmentation. As discussed above, the operator/surgeon may adjust the segmentation as needed. If the segmentation is not accepted, control continues to 832 to receive correction from the operator and return to 824.

If the segmentation is accepted, control continues to 836 to perform surgical simulation on the segmented, three-dimensional model. Control continues to 840 to determine if the operator has accepted the simulation. If no, control continues to 844 to receive correction from the operator and return to 836. Otherwise, control continues to 848 to model implants using the surgical simulation. Control proceeds to 852 to determine if operator has accepted the modeled implants. If no, control continues to 856 to receive correction from the operator and return to 848. Otherwise, control proceeds to 860 to perform validation of the modeled implants.

At 864, control determines if the operator has accepted the validation of the implants. If no, control continues to 868 to receive correction from the operator and return to 860. Otherwise, control proceeds to 872 to generate and transmit digital files for fabrication of the implant. Then, control ends.

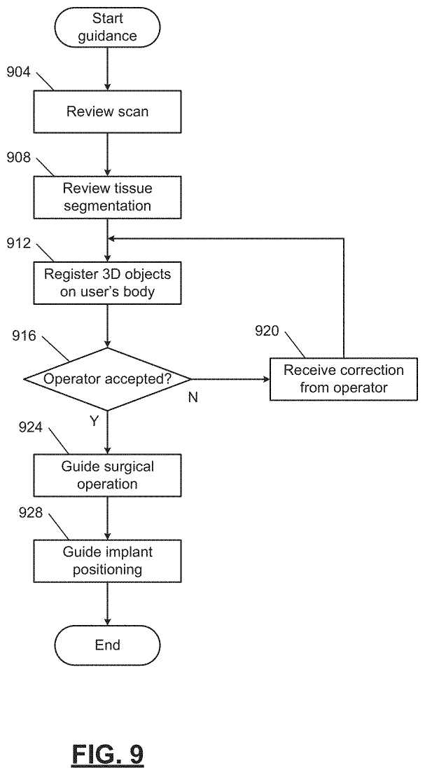

FIG. 9 is another example flowchart describing the specific implementation steps of surgical guidance (G1 to G4). Control begins guidance by, for example, and operator selecting a start button on a user interface. At 904, control reviews the scan (or other medical imaging file) that was used for the guidance. At 908, control reviews the tissue segmentation. Control continues to 912 to register three-dimensional objects on the user's body. Control proceeds to 916 to determine if the operator accepted the registration of the three-dimensional objects on the user's body. If no, control continues to 920 to receive correction from the operator and return to 912. Otherwise, control proceeds to 924 to perform guidance of the surgical operation. Control continues to 928 to perform guidance of implant positioning.

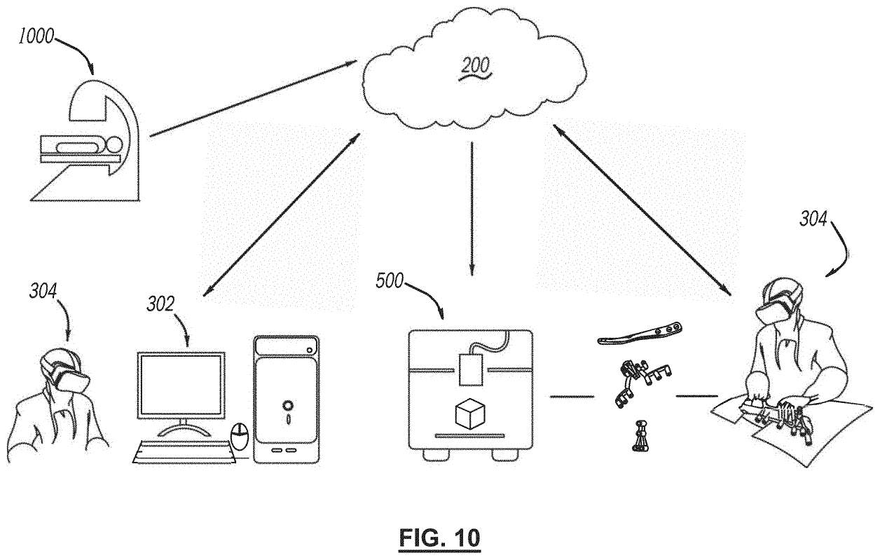

FIG. 10 is a high level depiction of an implementation of the system 100. As described throughout the present disclosure, the system 100 is implemented via a distributed communication network or data processing infrastructure 200 including a processor and memory. The data processing infrastructure 200 receives scans from a scanning device 1000, such as a CT scan or other medical imaging file, and generates surgical guidance and digital implant files. The surgical guidance can be forwarded to a plurality of users or devices, such as the computer terminal module 302 and augmented reality module 304 (such as a streaming headset. Further, the digital implant files, instructing construction of implants (developed using the surgical guidance function applied to a particular user) can be transmitted to the manufacturing facility module 500 that can construct the implant at the facility.

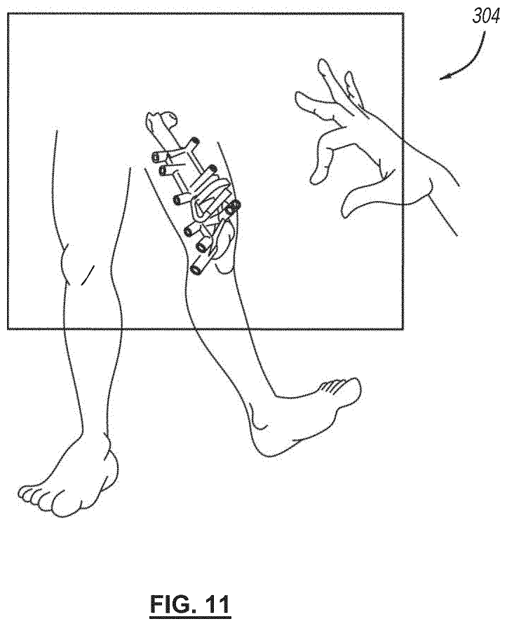

FIG. 11 is a graphical depiction of an operator view of a guided surgical operation using the system 100. In particular, the augmented reality module 304 is shown in a mixed reality view, depicting a surgical guide (which also could be an implant) on the particular user to assist in guiding the operator (shown as a hand).

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

What is claimed is:

1. A surgical support method, comprising:
obtaining raw medical imaging data corresponding to a user from a remote computing architecture;
reconstructing a digital model from the obtained raw medical imaging data, wherein the digital model is two-dimensional or three-dimensional;
determining at least one pathology based on the digital model;
obtaining a sequence of surgical acts based on the at least one pathology;
generating a plurality of three-dimensional scenes by applying the sequence of surgical acts to the digital model;
simulating a virtual performance of the sequence of surgical acts on the digital model of the user using the generated plurality of three-dimensional scenes; and
displaying, on a display, at least one of: the reconstructed digital model, the at least one pathology, the generated plurality of three-dimensional scenes, and the simulated virtual performance.

2. The surgical support method of claim 1 further comprising:
extracting data of interest from the reconstructed digital model; and
determining the at least one pathology based on the extracted data of interest.

3. The surgical support method of claim 1 further comprising:
projecting the plurality of three-dimensional scenes onto the user to guide an operator, wherein the projecting includes holographic projection.

4. The surgical support method of claim 3 wherein the holographic projection can be manipulated by the operator or can be positioned on the user.

5. The surgical support method of claim 3, wherein the generated plurality of three-dimensional scenes or the simulated virtual performance is displayed in a collaborative mode to remotely assist the operator to perform a corresponding procedure requiring multiple assessments or to train the operator in an observational mode.

6. The surgical support method of claim 1 further comprising:
implementing artificial intelligence and/or a simulation of physical systems by numerical mathematic modeling to model the simulated virtual performance.

7. The surgical support method of claim 1 further comprising:
generating device digital models of at least one of: anatomical elements, implantable medical devices, and ancillary instrumentation corresponding to the sequence of surgical acts, wherein the device digital models include three-dimensional objects based on the reconstructed digital model or data of interest extracted from the reconstructed digital model.

8. The surgical support method of claim 7 wherein the anatomical elements, the implantable medical devices, or the ancillary instrumentation are based on anatomy of the user.

9. The surgical support method of claim 7 further comprising:
projecting the device digital models onto the user to guide an operator, wherein the projecting includes holographic projection.

10. The surgical support method of claim 1 further comprising:
in response to a structured surgical planning of the user ending, generating device digital models of at least one of: anatomical elements, implantable medical devices, and ancillary instrumentation.

11. The surgical support method of claim 10 further comprising:
generating a digital file based on the device digital models; and
transmitting the generated digital file to a manufacturing facility to create a three-dimensional model using the generated digital file.

12. A surgical support system, comprising:
at least one processor and
a memory coupled to the at least one processor,
wherein the memory stores:
a database including a set of raw medical imaging data and a set of surgical acts, wherein each set of raw medical imaging data corresponds to a user; and
instructions executed by the at least one processor and wherein the instructions include:
obtaining raw medical imaging data corresponding to a user from the database;
reconstructing a digital model from the obtained raw medical imaging data, wherein the digital model is two-dimensional or three-dimensional;
extracting data of interest from the reconstructed digital model;
determining at least one pathology based on the extracted data of interest;
obtaining a sequence of surgical acts based on the at least one pathology from the database;
generating a plurality of three-dimensional scenes by applying the sequence of surgical acts to the extracted data of interest;
simulating a virtual performance of the sequence of surgical acts on the digital model of the user using the generated plurality of three-dimensional scenes; and
displaying, on a display, at least one of: the reconstructed digital model, the at least one pathology, the generated plurality of three-dimensional scenes, and the simulated virtual performance.

13. The surgical support system of claim 12 wherein the instructions include:
projecting the plurality of three-dimensional scenes onto the user to guide an operator, wherein the projecting includes holographic projection.

14. The surgical support system of claim 13 wherein the holographic projection can be manipulated by the operator or can be positioned on the user.

15. The surgical support system of claim 13, wherein the generated plurality of three-dimensional scenes or the simulated virtual performance is displayed in a collaborative mode to remotely assist the operator to perform a corresponding procedure requiring multiple assessments or to train the operator in an observational mode.

16. The surgical support system of claim 12 wherein the instructions include:
implementing artificial intelligence and/or a simulation of physical systems by numerical mathematic modeling to model the simulated virtual performance.

17. The surgical support system of claim 12 wherein the instructions include:
generating device digital models of at least one of: anatomical elements, implantable medical devices, and ancillary instrumentation corresponding to the sequence of surgical acts, wherein the device digital models include three-dimensional objects based on the reconstructed digital model or the extracted data of interest.

18. The surgical support system of claim 17 wherein the anatomical elements, the implantable medical devices, or the ancillary instrumentation are based on anatomy of the user.

19. The surgical support system of claim 17 wherein the instructions include:
projecting the device digital models onto the user to guide an operator, wherein the projecting includes holographic projection.

20. Computer software, stored in a non-transitory computer-readable memory, the software comprising:
instructions receiving medical imaging data corresponding to a user from a database;
instructions creating a digital model from the medical imaging data;
instructions using data of interest from the digital model;
instructions determining at least one pathology based on the data of interest;
instructions receiving a sequence of surgical acts based on the at least one pathology;
instructions generating a visual image by applying the sequence of surgical acts to the data of interest;
instructions simulating a virtual performance of the sequence of surgical acts on the digital model of the user using the generated images;
instructions projecting, via a headset, the generated images to guide an operator; and
instructions displaying, on a display of the headset, at least one of: the digital model, the at least one pathology, the generated images, and the simulated virtual performance.

21. The computer software of claim 20 wherein projecting includes projecting a holographic projection, and the holographic projection can be manipulated by the operator or can be positioned on the user.

22. The computer software of claim 20, wherein the generated images or the simulated virtual performance is displayed in a collaborative mode to remotely assist the operator to perform a corresponding procedure requiring multiple assessments or to train the operator in an observational mode.

23. The computer software of claim 20 further comprising:
instructions implementing artificial intelligence and/or a simulation of physical systems by numerical mathematic modeling to model the simulated virtual performance.

24. The computer software of claim 20 further comprising:
instructions generating device digital models of at least one of: anatomical elements, implantable medical devices, and ancillary instrumentation corresponding to the sequence of surgical acts, wherein the device digital models include three-dimensional objects based on the reconstructed digital model or the data of interest.

25. The computer software of claim 24 wherein the anatomical elements, the implantable medical devices, or the ancillary instrumentation are based on anatomy of the user.

26. The computer software of claim 24 further comprising:
instructions projecting the device digital models onto the user to guide the operator, wherein the projecting includes holographic projection.

27. A method of using for surgical preparation and surgery, the method comprising:
obtaining medical imaging data corresponding to a user;
constructing a digital model from the obtained medical imaging data;
selecting a sequence of surgical acts from a database;
generating a plurality of scenes by applying the sequence of surgical acts to the digital model;
simulating a virtual performance of the sequence of surgical acts on the digital model of the user using the generated scenes;
displaying at least one of: the reconstructed digital model, the generated plurality of scenes, and the simulated virtual performance; and
showing the plurality of scenes to guide surgical preparation and surgery associated with the user.

28. The method of claim 27 further comprising:
extracting data of interest from the reconstructed digital model; and
determining at least one pathology based on the extracted data of interest.

29. The method of claim 27 wherein showing includes projecting a holographic projection, and the holographic projection can be manipulated by the operator or can be positioned on the user.

30. The method of claim 27 further comprising:
implementing artificial intelligence and/or a simulation of physical systems by numerical mathematic modeling to model the simulated virtual performance.
